(19) United States
(12) Patent Application Publication
(10) Pub. No.: US 2026/0080983 A1
(43) Pub. Date: Mar. 19, 2026

(54) SYSTEMS AND METHODS FOR DEIDENTIFIED DATA PROCESSING

(71) Applicant: Atropos Health, Inc., New York, NY (US)

(72) Inventors: Vladimir Polony, Novato, CA (US); Rehan N. Chawdry, Dublin, OH (US); Saurabh Gombar, San Diego, CA (US); Neil M. Sanghavi, Dublin, OH (US); Brigham Hyde, Purchase, NY (US)

(21) Appl. No.: 19/328,858

(22) Filed: Sep. 15, 2025

Related U.S. Application Data
(60) Provisional application No. 63/694,449, filed on Sep. 13, 2024.

(51) Int. Cl. G16H 10/20; G06F 16/245

(52) U.S. Cl. CPC G16H 10/20; G06F 16/245

(57) ABSTRACT

Systems and methods for deidentified data processing are provided herein. Deidentified data processing techniques can include accessing data from one or more worker nodes and assembling accessed data into patient timeline vectors. Worker nodes can provide sub-vectors to a primary node, which can assemble sub-vectors across multiple worker nodes to build patient timeline vectors that include data from multiple sources. Data provided by worker nodes can be deidentified, and subjects across different worker nodes can be matched based on a hash or other unique identifier. In some implementations, worker nodes provide compressed data tables with subject data, and a primary node reconstructs tables from the compressed format. In some implementations, only relative dates are utilized to better preserve patient privacy.

### CROSS-REFERENCE TO RELATED APPLICATIONS

This application claims the benefit of priority to U.S. Provisional Application No. 63/694,449, filed Sep. 13, 2024, titled “SYSTEMS AND METHODS FOR DEIDENTIFIED DATA PROCESSING,” the contents of which are incorporated by reference as if set forth fully herein. Any and all applications for which a foreign or domestic priority claim is identified in the Application Data Sheet as filed with the present application are hereby incorporated by reference under 37 CFR 1.57 for all purposes and for all that they contain.

### TECHNICAL FIELD

Implementations of the present disclosure related to deidentified data processing. Some implementations enable analysis of data across multiple sources in a privacy-preserving manner. Some implementations relate to identifying and processing deidentified data across multiple sources in order to perform medical studies, such as retrospective studies, based on clinical data, prescription data, claims data, and other relevant data.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Clinical trials are a powerful tool for establishing cause-and-effect relationships due to their rigorous design. By prospectively collecting data, using randomization and control groups, and conducting blind studies, clinical trials can minimize bias and enable researchers to isolate the actual effect of an intervention with a high degree of confidence. However, there are also significant limitations to clinical trials. For example, sample sizes can be relatively small, and eligibility criteria can result in a study population that doesn't reflect the diversity of real-world patients, potentially limiting the generalizability of a trial. Furthermore, clinical trials can be expensive and time-consuming, making them impractical for many applications.

Retrospective studies provide greater efficiency as researchers can analyze vast amounts of existing data to identify potential associations, generate new hypotheses, and so forth. This can be particularly powerful when investing outcomes over long time periods (e.g., over many years), when investigating rare diseases, and other circumstances where clinical trials are impractical or economically infeasible. However, retrospective studies can have significant limitations. For example, researchers have no control over how the original data was collected, leading to a significant risk of inaccuracies, missing information, and confounding variables that can distort results. An individual patient's medical history may be dispersed across a wide variety of systems, including electronic health record systems, pharmacy systems, insurance claim systems, and so forth, presenting a significant challenge for creating a complete view of a patient's medical history.

### SUMMARY

The approaches described herein each have several aspects, no single one of which is solely responsible for its desirable attributes. Without limiting the scope of this disclosure, several non-limiting features will now be described briefly.

In some embodiments, the techniques described herein relate to a computer-implemented method for deidentified data processing, the computer-implemented method including: accessing, by a primary node, a query, wherein the query includes a plurality of population conditions for defining a cohort to be included in a retrospective medical study; accessing a plurality of patient feature set matrices, wherein each patient feature set matrix of the plurality of patient feature set matrices corresponds to a worker node of the plurality of worker nodes, wherein each patient feature set matrix indicates data present on or accessible by the corresponding worker node, wherein each patient feature set matrix indicates which patients have data corresponding to a plurality of features; determining, based on the plurality of population conditions and the plurality of patient feature set matrices, a set of target worker nodes and a set of patients, wherein the target worker nodes of the set of target worker nodes are selected from the plurality of worker nodes, wherein each target worker node of the set of target worker nodes is a node that is determined to possibly have data responsive to the query, wherein the set of patients is selected by identifying patients with data corresponding to a population condition; causing execution of the worker node queries on the identified set of target worker nodes; accessing results returned by the target worker nodes of the set of target worker nodes; generating a dataset including patient timeline objects using the results, wherein the patient timeline objects indicate a presence of a feature at a time, wherein the time is measured relative to a reference date; and making the dataset available for use.

In some embodiments, the techniques described herein relate to a computer-implemented method for deidentified data processing, the computer-implemented method including: accessing, by a primary node, a query, wherein the query includes a plurality of population conditions for defining a cohort for a retrospective medical study; determining, by the primary node using one or more feature set matrices, a first set of patients, wherein the first set of patients includes patients who could be included in the cohort based on one or more population conditions of the plurality of population conditions; generating, by the primary node, one or more requests for data from one or more worker nodes, wherein the one or more requests are configured to request data associated with the first set of patients from the one or more worker nodes; transmitting, by the primary node, the one or more requests to the one or more worker nodes; accessing, by the primary node, a plurality of responses from a plurality of worker nodes, the responses generated by the worker nodes in response to executing the one or more generated queries; generating, by the primary node, a dataset based on the plurality of responses, wherein the dataset includes a plurality of patient timeline vectors for a plurality of cohort patients, wherein at least one of the plurality of patient timeline vectors is generated by combining a first response from a first worker node and a second response from a second worker node that is different from the first worker node.

In some embodiments, the techniques described herein relate to a system including: one or more hardware processors; and a non-transitory computer-readable storage medium having instructions stored thereon that, when executed by the one or more hardware processors, cause the system to: generate a query, wherein the query includes a plurality of population conditions for defining a cohort for a retrospective medical study; determine, using one or more feature set matrices, a first set of patients, wherein the first set of patients includes patients who could be included in the cohort based on one or more population conditions of the plurality of population conditions; generate one or more requests for data from one or more worker nodes, wherein the one or more requests are configured to request data associated with the first set of patients from the one or more worker nodes; transmit the one or more requests to the one or more worker nodes; access a plurality of responses from a plurality of worker nodes, the responses generated by the worker nodes in response to executing the one or more generated queries; generate a dataset based on the plurality of responses, wherein the dataset includes a plurality of patient timeline vectors for a plurality of cohort patients, wherein at least one of the plurality of patient timeline vectors is generated by combining a first response from a first worker node and a second response from a second worker node that is different from the first worker node.

### BRIEF DESCRIPTION OF THE DRAWINGS

Detailed descriptions of implementations of the present invention will be described and explained through the use of the accompanying drawings.

FIG. 1 is a diagram that illustrates an example environment in which the approaches described herein can be carried out according to some implementations.

FIG. 2 is a flowchart that illustrates an example data retrieval and delivery process according to some implementations.

FIG. 3 is a flowchart that illustrates an example process for retrieving data according to some implementations.

FIG. 4 is a drawing that illustrates an example of a multimode data artifact according to some implementations.

FIG. 5 is a drawing that illustrates an example of data at remote nodes (also referred to as distributed nodes) and a final multinode dataset at a primary node according to some implementations.

FIG. 6 is a flowchart that illustrates an example of generating patient tokens and deidentifying patient data according to some implementations.

FIG. 7 is a flowchart that illustrates an example of adding a new node to a network of nodes.

FIG. 8 is a drawing that illustrates an example patient feature matrix according to some implementations.

FIG. 9 is a flowchart that illustrates a process for retrieving and analyzing patient data according to some implementations.

FIG. 10 is a diagram that schematically illustrates a patient timeline vector according to some implementations.

FIGS. 11A and 11B show examples of interval-encoded data according to some implementations.

FIG. 12 is a block diagram that illustrates various components of a system according to the present disclosure.

FIG. 13 illustrates an example process for generating and using patient timeline vectors according to some implementations.

FIG. 14 is a block diagram that illustrates an example system and related features according to some implementations.

FIG. 15 is a flowchart that illustrates an example process for retrieving and analyzing patient data according to some implementations.

FIG. 16 illustrates an example process for generating patient timeline vectors according to some implementations.

FIG. 17 schematically illustrates the generation of a patient timeline vector according to some implementations.

FIG. 18 is a flowchart that illustrates an example process for identifying datasets that contain data for patients in the study population and extracting information for those patients according to some implementations.

FIG. 19 is a flowchart that illustrates an example process for retrieving and processing patient information according to some implementations.

FIG. 20 is a flowchart that illustrates an example process for identifying patients who match population criteria according to some implementations.

FIG. 21 is a drawing that illustrates a Venn diagram of patients matching one or more population criteria.

FIG. 22 illustrates an iterative approach to identifying patients matching study criteria according to some implementations.

FIG. 23 schematically illustrates routing tables that can be used to locate patient records according to some implementations.

FIG. 24A illustrates an example process for sharing a reference date among worker nodes according to some implementations.

FIG. 24B illustrates a decentralized approach to sharing a reference date among worker nodes according to some implementations.

FIG. 25 is a flowchart that illustrates an example data retrieval process according to some implementations.

FIG. 26 is a block diagram depicting an embodiment of a computer hardware system configured to run software for implementing one or more of the systems and methods described herein.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION

Although several implementations, embodiments, examples, and illustrations are disclosed below, it will be understood by those of ordinary skill in the art that the scope of the present disclosure extends beyond the specifically disclosed implementations, embodiments, examples, and illustrations and includes other uses of the inventions and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of certain specific implementations. In addition, implementations can comprise several novel features, and no single feature is necessarily essential or solely responsible for its desirable attributes.

Electronic Health Records (EHR) and other electronic medical data storage have had a marked impact on the way healthcare data is collected, stored, and utilized. EHR systems can store a wealth of patient information, such as medical history, diagnoses, medications, treatment plans, immunization dates, allergies, radiology images, and laboratory test results. This wealth of data presents an opportunity for researchers to conduct studies that can lead to significant advancements in medical science, healthcare delivery, public health policy, and so forth.

Unlike traditional clinical trials, which often involve a limited number of participants and controlled conditions, EHR data, prescription claims data, pharmacy data, and so forth encompass a broad spectrum of real-world patient experiences, providing a greater volume and depth of data than would ordinarily be possible in clinical trials. This allows researchers to study a wide range of conditions and treatments across large and/or diverse populations, which can lead to more generalizable findings, can be used to answer questions that have not been subjected to clinical trials, and so forth. For instance, researchers can identify patterns and trends in disease prevalence, treatment outcomes, and healthcare utilization, which can inform better clinical practices, health policies, and so forth.

EHR data can enable longitudinal studies, where researchers can track patient outcomes over extended periods, providing a significant differentiator over clinical trials. For example, clinical trial phases typically last from about a year to about four years, thus limiting the time period that can be considered and making it difficult or impossible to detect long-term outcomes. Using EHR data, patients can be tracked over many years or decades. This is particularly valuable for understanding the long-term effects of treatments, the progression of chronic diseases, and the impact of preventive measures. Moreover, studies can be conducted without necessarily requiring patients to actively participate in a trial, as data can be obtained from health records generated from routine medical care. Longitudinal data can reveal insights that are not apparent in short-term studies, such as the development of comorbidities or the effectiveness of lifestyle interventions over time. By analyzing EHR data, researchers can also identify early warning signs of adverse events or complications, leading to improved patient monitoring and timely interventions.

However, utilizing EHR data for research also presents several challenges. One of the primary concerns is data quality and consistency. EHR systems are designed primarily for clinical use, and the data entered by healthcare providers may vary in completeness and accuracy. Inconsistent coding practices, missing data, and variations in how information is recorded can complicate data analysis and potentially lead to incorrect conclusions. Thus, EHR and other data can be cleaned, normalized, etc., before being used for studies.

Another significant challenge is ensuring patient privacy and data security. EHR and other medical data contain sensitive personal information, which could potentially be leaked or otherwise improperly disclosed if not handled carefully, potentially exposing researchers, medical providers, etc., to legal risk, financial risk, reputational risk, and so forth. Protecting patient data often involves deidentifying data, obtaining approvals from institutional review boards (IRBs), implementing secure data storage and access protocols, and so forth.

Patient data deidentification can involve removing or altering personal information from datasets. This process can reduce the likelihood that data can be traced back to specific individuals. There are several methods for deidentifying patient data, including safe harbor and statistical deidentification.

The safe harbor method is relatively straightforward. In the safe harbor method, specific identifiers are removed or obfuscated in the data. HIPAA outlines types of information to be removed from data to meet safe harbor standards, including names, geographic information (e.g., geographic information more fine-grained than state-level), dates directly related to an individual (e.g., date of birth, admission date, discharge date, etc.) (aside from year), phone numbers, fax numbers, email addresses, social security numbers, medical record numbers, health plan beneficiary numbers, account numbers, certificate/license numbers, vehicle identifiers, device identifiers, web URLs, IP addresses, biometric identifiers such as fingerprints and voiceprints, full-face photographs, or any other unique identifying number, characteristic, or code.

By eliminating these identifiers, the data is considered deidentified under the safe harbor method. This method is relatively simple to implement and provides a clear, standardized approach to deidentification. However, the loss of certain data can make conducting certain studies difficult or impossible. For example, studies that depend on time can be impossible if date information is removed. Moreover, adhering to safe harbor standards can make it difficult or impossible to combine patient data across datasets. For example, in some cases, it can be desirable to combine multiple datasets, such as datasets from multiple healthcare providers or data from different types of sources, such as medical records, prescription records, claims records, and so forth. However, if each dataset individually follows safe harbor methods, there may be no way to link records for a patient that appears in multiple datasets.

Statistical deidentification involves using statistical principles to ensure that the risk of re-identifying individuals in the dataset is very low. Unlike the safe harbor method, which relies on a predefined list of identifiers, statistical deidentification assesses the data as a whole. Experts apply various techniques to reduce the likelihood of re-identification, including data masking, pseudonymization, data perturbation, aggregation, suppression, and so forth. Data masking involves altering data in a way that conceals the original information. For example, replacing real names, dates of birth, etc., with a hash or other identifier that does not indicate a patient's identity. Pseudonymization replaces private identifiers with fake identifiers or pseudonyms. While the data can still be analyzed, the pseudonyms cannot be easily traced back to the original individuals without additional information. Data perturbation involves making small changes to the data to prevent re-identification. For example, adding random noise to numerical data or swapping data points between individuals. While data perturbation can be a useful tool, care must be taken to ensure that modifications to the data do not render it unfit for research purposes. Aggregation involves combining data from multiple individuals into summary statistics or larger groups to prevent the identification of specific individuals. Suppression involves removing certain data points that are too sensitive or unique, which could lead to re-identification.

Statistical deidentification is generally more flexible than safe harbor and can be tailored to the specific characteristics of the dataset, making it suitable for a wider range of applications. However, significant expertise in data science and statistics may be needed to ensure that statistical deidentification processes are effective.

There are several limitations to safe harbor and statistical deidentification. For example, safe harbor typically involves the removal of dates, which can make time-based questions difficult or impossible to answer. Statistical deidentification typically removes the most unique aspects of a patient's medical records, such as genetic results, a patient having taken a drug once, etc. As a result, it can be difficult or impossible to answer questions relating to more unique aspects of patients.

Combining health data from multiple sources can significantly enhance the depth and breadth of research, providing a more comprehensive understanding of health and disease. By integrating data from various healthcare providers, insurance companies, public health databases, pharmacies, wearable devices, mobile health apps, and so forth, researchers can create a richer dataset that captures a wider array of health information. Combining data can enable more robust analyses, for example, enabling the identification of complex interactions between genetic, environmental, behavioral, medical treatment, social factors, and so forth that influence health outcomes. For example, combining clinical data with social determinants of health data can help identify disparities in healthcare access and outcomes. As another example, combined EHR data and pharmacy data or pharmacy claims data can allow a researcher to consider patient compliance and can provide a more objective measure of patient compliance than, for example, reliance on a patient's self-reported compliance. For example, a physician can prescribe a medication to a patient, and pharmacy or pharmacy claims data can indicate whether or not the patient filled the prescription, refilled the prescription, refilled the prescription on time, and so forth. This information can be significant for researchers. For example, a patient who was prescribed a treatment but never filled the prescription can be excluded from a study or more properly categorized as not having received the treatment, rather than categorized into a treatment group simply because the treatment was prescribed. As another example, a study can have a third group that includes individuals whose records indicate that they sporadically took the prescription, which can potentially reveal insights into the effects of poor compliance.

While many studies may utilize more traditional sources of data, other sources may also be used in some studies. For example, the growth of the wearable industry (e.g., smartwatches, wearable pulse rate monitors, wearable glucose monitors, etc.), presents the opportunity to analyze a wealth of data from a broad spectrum of individuals, with data collected much more frequently than in the past, when certain measurements might only be recorded at follow up visits, hospitalizations, and so forth. In some cases, such data may be uploaded or otherwise stored in or accessible by an EHR system, although in other cases, such data may be stored in other repositories.

The process of integrating health data from multiple sources presents various challenges. For example, there can be interoperability challenges as different systems often use varying formats, terminologies, coding systems, and standards. Additionally, maintaining data quality and consistency across sources can be a significant challenge, as discrepancies and inaccuracies can compromise the validity of research findings. Records may be incomplete, contain errors, and so forth. For example, there can be scrivener's errors in patient identifying data, such as name, date of birth, etc., which can make it difficult to match patients across datasets, patient histories may be incomplete or inaccurate, and so forth. Various techniques can be used to address such errors. For example, rather than using hashing methods such as MD5 or SHA to generate deidentified patient identifiers, other hashing techniques that generate similar hashes for similar, but not identical, inputs can be used. Such hashes can be compared (for example, using Levenshtein distance or other metrics) to determine if two hashes likely correspond to the same patient.

As described herein with respect to single datasets, patient privacy is a significant concern. Combining data from multiple sources can increase the risk of re-identification. For example, if a first dataset and a second dataset are each statistically deidentified, a dataset that combines the first and second datasets may not be statistically deidentified, as the combined information may be sufficient to identify individual patients.

While deidentification techniques can protect patient privacy by removing or altering personal identifiers, the complexity of deidentification can increase when data from different sources is merged. For instance, even if individual datasets are deidentified, combining them can sometimes reintroduce identifiable information through data linkage. This issue, known as the mosaic effect, occurs when disparate pieces of deidentified data are combined in a way that allows for the re-identification of individuals. Thus, there can be a need to go through statistical deidentification processes again for a combined dataset, which can take a significant amount of time, have significant costs, and so forth. As an example, consider an individual with a rare disease who lives in Minnesota. If one dataset indicates that the individual lives in Minnesota but does not indicate the rare disease, while another dataset indicates that the individual has the rare disease but not that the individual lives in Minnesota, it may be difficult or impossible to determine the identity of the patient from only one of the datasets. If these two datasets are combined, however, the individual may no longer be deidentified, as few people with the rare disease also live in Minnesota.

Moreover, the effectiveness of deidentification techniques can vary depending on the nature of the data and the computer-implementated methods used. Simple techniques like removing direct identifiers (e.g., names, Social Security numbers) may not be sufficient when dealing with complex datasets that include indirect identifiers (e.g., dates of service, geographic locations). Techniques such as data masking, pseudonymization, and data perturbation can help, but they require careful implementation and ongoing evaluation to ensure they remain effective and do not compromise research results. Additionally, the risk of re-identification can increase over time as new data sources become available, patient histories become longer, and computational methods for data analysis advance.

As described herein, balancing the need for data utility with the need to protect patient privacy can present significant challenges. Overly aggressive deidentification can strip the data of valuable information, rendering it less useful for research purposes. For example, removing all geographic information might protect privacy but also eliminate the ability to study regional variations in health outcomes. Stripping date information can make it difficult or impossible to track outcomes over time. Thus, there is a need for approaches that can allow for the use of various datasets while maintaining patient privacy in a manner that does not overly impede researchers' ability to use the data.

In conventional approaches, when data from multiple sources is used in a study, the data is combined into a single large dataset or several smaller datasets, and it is assumed that a single system or node has access to an entire record for a patient. However, this presents several challenges. For example, different organizations may own or control different datasets, and data for a single patient may be spread across multiple datasets owned by different medical providers, pharmacies, insurance providers, etc. Combining datasets can require that organizations relinquish control of their data by making full datasets (or large portions of datasets) available for combining with other datasets. This can present a significant risk as the organization loses control over data, potentially presenting risks for patient privacy and impacting the organization's ability to monetize its data in the future. Moreover, patients may be hesitant to authorize such use of their data if they perceive a significant risk that their private medical information may be shared in ways that could compromise their privacy.

Advantageously, some approaches herein can be used to search for and retrieve data across multiple sources, without requiring that those sources hand over full access to their datasets. For example, according to some implementations, queries can be executed against individual datasets and later combined. In some implementations, combining data can be done in a privacy-preserving manner that does not require deidentification processes to be repeated.

As described herein, conventional data analysis approaches expect that all data for a single patient be located in a single database, node, etc. However, this presents significant challenges in the real world, where patients often visit multiple providers, pharmacies, and so forth, resulting in patient data being distributed across multiple datasets. It can be significant to be able to have a single patient split across multiple datasets, to avoid creating a single unified dataset, etc. In some cases, data for a particular patient may not be included in every dataset. In some implementations, queries are executed only against datasets that contain information for patients of interest.

As described herein, combining datasets into a single unified dataset can break expert determinations regarding deidentification and trigger a need to re-deidentify data. This can mean that, typically, to create a full patient record from multiple data sources, all data is aggregated into a single dataset, and then deidentification is carried out on the aggregated dataset.

In some implementations, the approaches herein can enable analysis of patient data that is distributed across datasets without the need to create a full patient record in a single location or system. In some implementations, the approaches herein can be used to create a date-removed or safe-harbored interval encoding that can act as a single timeline for a patient. The approaches herein can, in some implementations, avoid recombination and the issues associated with combining data, or can avoid certain issues associated with recombination, for example, because combining data can happen at an interval level, rather than a record level, as described in more detail herein. That is, an interval timeline encoding can be generated for a patient, and the timeline does not need to include actual dates such as dates of service, birthdates, diagnosis dates, prescription fill dates, and so forth. The approaches described herein can, in some implementations, allow data from different datasets to be combined in a manner that does not trigger a need for deidentification of the combined data. In some implementations, there can be some data combination while avoiding the combination of sensitive, potentially identifying data.

Advantageously, according to some implementations, individual datasets (e.g., individual deidentified datasets) can include temporal information, such as specific dates. As described herein, individual datasets can be processed by worker nodes that do not have visibility into other datasets. Thus, a query for medical records can include a date parameter that can be used when retrieving matching records. The worker nodes can strip out dates and generate an interval-encoded representation that can be used for further processing. For example, in some implementations, the interval-encoded representations are provided to a primary node that combines the representations into a patient timeline vector. In some implementations, the interval-encoded representations can be provided to an analysis module, which can perform analysis using the interval-encoded representations.

In some implementations, the analysis module can combine representations as needed for analysis. In some implementations, patient timeline vectors can be stored in non-volatile memory. In some implementations, patient timeline vectors are not stored in non-volatile memory, and are instead stored only in volatile memory (e.g., RAM). Not storing patient timeline vectors in non-volatile memory can have certain advantages, such as reducing the risk that a nefarious actor could obtain a copy of the patient timeline vectors. In either case, any combined data or patient timeline vectors can be deleted by a primary node or analysis module after analysis is complete or when the information is otherwise no longer needed, or after a threshold period of time has expired (e.g., after a time to live (TTL)).

In some implementations, patients can be identified using a token identifier or hash. Typically, token identifiers are based on information such as the patient's name, gender, date of birth, location, etc. In some implementations, a token identifier can be randomly generated. However, randomly-generated identifiers can make it difficult to ensure that the same patient has the same identifier across datasets. Thus, it can be significant to generate tokens using information about the patient. Token identifiers can be shared across datasets, making it possible to join data for a single patient across different datasets, even when the datasets have been deidentified. Token identifiers typically, though not necessarily, use a one-way hash, which can prevent reconstruction of the underlying information used to generate the hash. Different information can be used when generating hashes, and the information used can result in higher or lower confidence that records across datasets correspond to the same patient. For example, a more relaxed approach that uses less data can result in more matches across datasets, but at the increased risk that patients with the same hash are not actually the same person. In some implementations, a system determines multiple hashes for a patient, and users can select more or less rigorous matching criteria depending upon desired confidence levels, target sample size, etc. For example, a hash can be based on name and date of birth; name, date of birth, and address; name, date of birth, and social security number; name, date of birth, social security number, and address; or any other combination of identifying information. The identifying information can be used as inputs when generating a hash or pseudonym for the patient.

The decentralized approaches herein can address certain challenges associated with decentralized data. For example, in conventional approaches, identifying patients who match a study population definition is fairly straightforward. For example, if all data is contained in a single database, searching the database to identify patients who match study population criteria can be straightforward. For example, consider a study population made up of diabetic males over age 55 who also have pancreatic cancer. A query for patients who match the study population criteria could be formulated as, for example, “sex=‘Male’ AND condition like ‘Diabetes’ AND age >=55 AND condition like ‘pancreatic cancer’.” However, if patient data is spread across multiple datasets, such a query might fail to return all relevant patients. For example, one dataset may indicate that a male patient over 55 has diabetes but may not indicate that the patient has cancer, while another dataset may indicate that the patient has pancreatic cancer but does not indicate that the patient also has diabetes. In some implementations, a system can be configured to identify patients matching any criteria for the study population definition and can combine those patients to identify a subset of patients that match all the study population criteria.

As an example, consider a population defined as ‘condition A AND condition B AND condition C.’ In some implementations, worker nodes can execute queries for ‘condition A OR condition B OR condition C.’ A node (e.g., a primary node) can combine results from multiple worker nodes to identify an intersection of patients, thereby determining which patients meet condition A, condition B, and condition C.

After patients in a population are identified, queries can be executed to obtain relevant health or other data for those patients. In some implementations, queries can be executed against every dataset (e.g., by every worker node in a set of worker nodes). However, executing queries against every dataset may be inefficient, as it is possible that not all datasets include information for the patients included in the study. Additionally, dataset owners or controllers may charge for access or limit the number of queries that can be executed to prevent or limit heavy loads on systems.

In some implementations, a system can be configured to map patient identifiers and datasets. In some implementations, each node of a set of nodes can have a complete copy of the mapping or can have access to a complete copy of the mapping, for example, access to a mapping stored on a primary node. In some implementations, the mapping can be distributed, in which each node has only a portion of the full mapping. In some implementations, mapping can utilize techniques such as finger tables or lists of nodes at various distances to allow easy, efficient identification of nodes that contain information for specific patients. For example, a finger table can contain information about other nodes at various distances in an identifier space, which can allow for efficient lookups. In some implementations, distances between nodes can be measured using, for example, an XOR metric. In some implementations, a system can implement Chord or Kademlia distributed hash table approaches, or adaptations thereof that are suited to looking up patient information across a number of datasets.

In some implementations, a primary node does not store identifiable data. For example, identifiable data can be located only on worker nodes, and the primary node may only have, for example, information about whether a worker node contains information about a particular condition or other factor in a study. For example, worker nodes can publish or otherwise provide feature sets to the primary node indicating what data is present on or accessible by the worker node. For example, a feature set could indicate that a worker node has information related to diabetes or contains medical images. In some implementations, the primary node stores a shared representation of how long it has been since a reference date, such as a patient's birthdate, but does not actually store the patient's birthdate or otherwise have access to the patient's date of birth.

In some implementations, the primary node does not retain data. For example, the primary node can process data in memory and discard the data once processing is complete.

While patient timeline sub-vectors and vectors can be used to assemble patient data in a privacy-preserving manner, in some cases it can be beneficial in some circumstances to transmit more complete patient records to the primary node. For example, for certain complex or evolving analyses, transferring raw patient data to the primary node can provide flexibility and lessen or even eliminate the need to carry out additional queries on the worker node(s). In some implementations, a worker node can send a table to the primary node containing patient data. The table can contain a subset of data for a patient or multiple patients, all data for a patient or multiple patients, or a combination of partial and full data. In some implementations, the worker node compresses the table and transmits the compressed representation to the primary node, and the primary node can reconstruct the table. In some implementations, a binary compressed representation is created by a worker node and transmitted to the primary node. Various algorithms can be used for compression, such as LZ-based algorithms, run-length encoding, etc. In some implementations, dictionary encoding, delta encoding, or bit-packing is used. In some implementations, the worker node sends a single table, but other configurations are possible. For example, a worker node may transmit a plurality of tables, either in a single compressed file or as multiple compressed files or data streams.

### Example Implementations

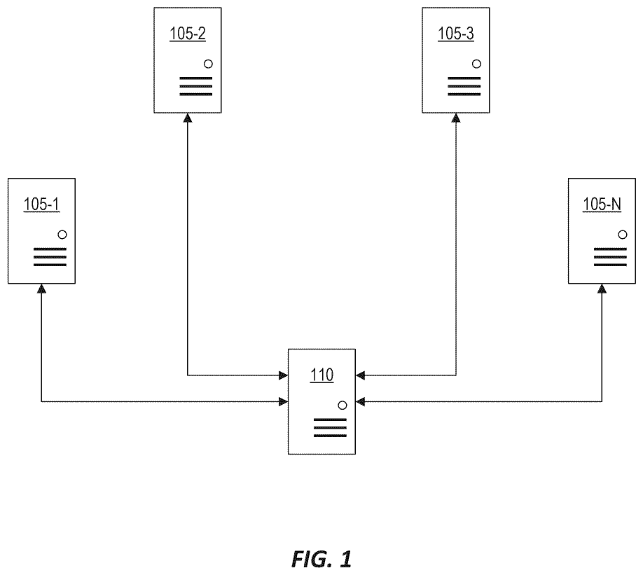

FIG. 1 is a diagram that illustrates an example environment in which the approaches described herein can be carried out according to some implementations. In FIG. 1, a primary node 110 is in communication with a plurality of worker nodes 105-1-105-N (collectively worker nodes 105 or individually worker node 105, also referred to as remote nodes). Each worker node 105 can include or can be connected to a data store (e.g., connected via a network). In some implementations, each of the primary node 110 and the worker nodes 105 run the same or similar software. That is, in some implementations, any worker node can be a primary node or any primary node can be a worker node. The role taken by a particular node can depend on what is being done, who it is being done by, etc. For example, if Organization A and Organization B both run nodes, when someone from Organization A accesses data, a node operated by or associated with Organization A can be the primary node, though this is not necessarily the case.

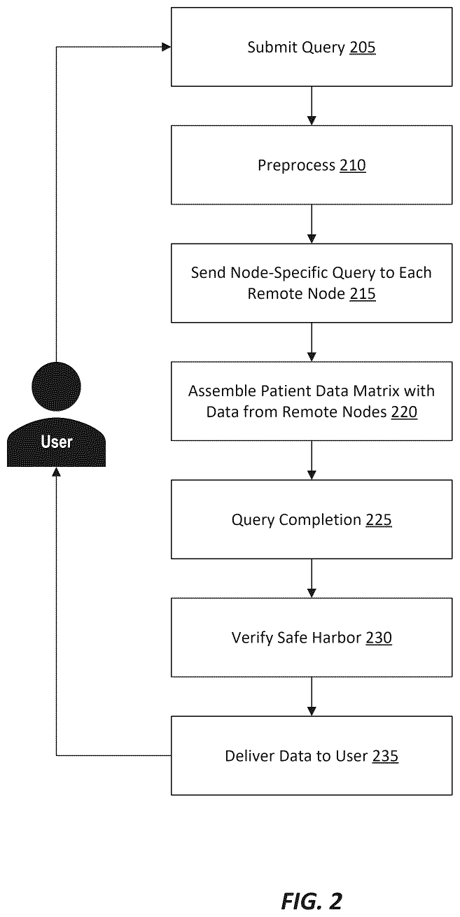

FIG. 2 is a flowchart that illustrates an example data retrieval and delivery process according to some implementations. At operation 205, a user can submit a question to a primary node. At operation 210, the primary node can preprocess the question received from the user to generate a query that describes the (deidentified) patients and features desired across an entire network of nodes. The master node can access a central patient/feature matrix (also referred to herein as a patient feature set or feature set) to determine which patients could possibly match the query. At operation 215, the primary node can send the query (or a node-specific query) to each eligible remote node (e.g., to each remote node that contains information for a potentially eligible patient). Each eligible node can execute the query to retrieve records responsive to the query. At operation 220, the system can receive data from the remote nodes and assemble aggregated data using the received data. The received data can include only data that is relevant to the query. The received data can be deidentified. For example, the received data can be limited to patient token (token id), birth year, and elapsed time (e.g., number of days) to one or more features of interest. As an example, if a patient born in 2013 was administered a vaccine on their first birthday, the received data can have a form such as “ID123, 2013, 365.” In some implementations, a remote node sends data only after verifying that no element of safe harbor is violated. At operation 225, the query can be complete. At operation 230, the primary node can verify that the assembled data meets safe harbor requirements. At operation 235, the system can provide a response to the user. The response can include, for example, the final results of performing a query on the aggregated data.

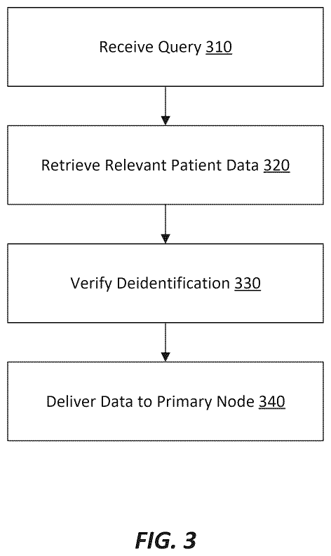

FIG. 3 is a flowchart that illustrates an example process for retrieving data according to some implementations. The process illustrated in FIG. 3 can be performed by a remote node. The process can be repeated for multiple worker nodes, which can execute queries to retrieve different (or at least partially different) data. At operation 310, a remote node can receive a query from a primary node. At operation 320, the remote node can retrieve relevant patient data based on the query. At operation 330, the remote node can verify that the retrieved data is deidentified or meets one or more deidentification standards. At operation 340, the remote node can deliver the data to the primary node, for example via a network connection.

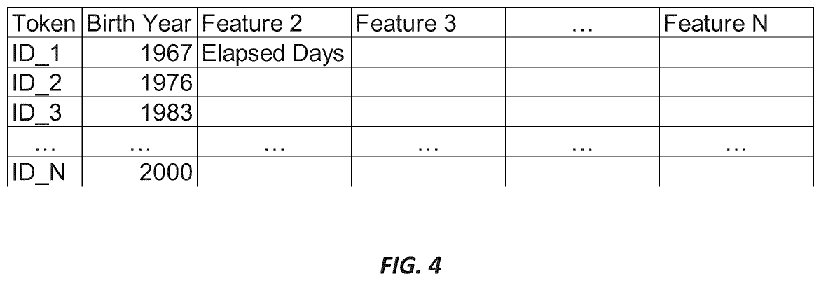

FIG. 4 is a drawing that illustrates an example of a multimode data artifact according to some implementations. The data artifact shows information for a variety of patients (identified by token IDs). In FIG. 4, the artifact shows birth year for each patient and features for each patient. In the example of FIG. 4, not all features are populated for all patients. The artifact can be an example of an artifact that is returned to a primary node in response to a query being executed on a remote node.

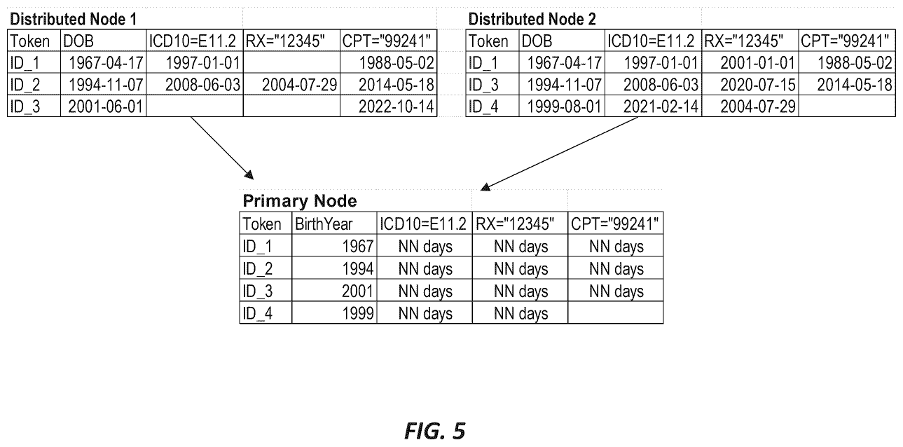

FIG. 5 is a drawing that illustrates an example of data at remote nodes (also referred to as distributed nodes) and a final multinode dataset at a primary node according to some implementations. The multinode dataset can be the result of combined the data at remote nodes. Multiple patients are represented, with values for various features, including particular ICD codes, RX codes, and CPT codes. As shown in FIG. 5, Distributed Node 1 has access to data for patients that is not available to Distributed Node 2. The combined set at the primary node includes information from Distributed Node 1 and Distributed Node 2. In the combined set, the dates of birth for the patients have been changed so that only birth year is indicated, thereby helping protect patient privacy and reduce the likelihood that the combined data can be deidentified. Similarly, the exact dates of diagnosis, prescription, and procedure have been replaced with a number of days that have elapsed from a reference date. The number of days that have elapsed can be days from the patient's date of birth or year of birth. Other reference dates are possible.

In some implementations, some date-based operations are not permitted in order to protect patient privacy. For example, examining a specific time period (such as the onset of the COVID-19 pandemic) could inadvertently expose approximate dates of birth for patients when combined with data such as age. A system can be configured to deny certain queries or fail to return certain data when there is an unacceptable risk of PII being exposed.

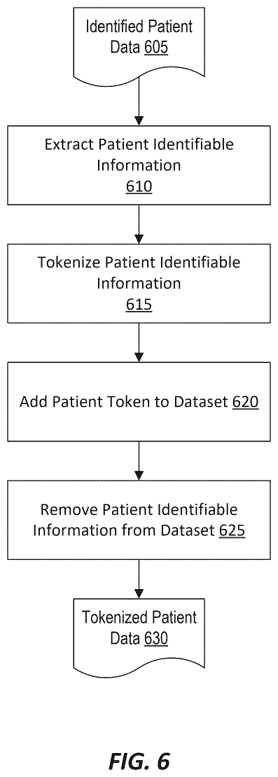

FIG. 6 is a flowchart that illustrates an example of generating patient tokens and deidentifying patient data according to some implementations. The patient tokens can be used to uniquely identify patients in one or more datasets. However, it may not be possible to readily determine the actual identity of particular patients based on the patient tokens. That is, the patient tokens can anonymously or pseudonymously identify patients. At operation 610, a system can extract patient identifiable information (e.g., name, date of birth) from identified patient data 605. At operation 615, the system can generate patient tokens using the patient identifiable information. At operation 620, the system can add the patient tokens to records in a dataset. At operation 625, the system can remove patient identifiable information from the dataset, resulting in tokenized patient data 630. In some implementations, the remote node may retain the patient identifiable information, though this information may not be passed to a primary node, as doing so could violate patient privacy rules.

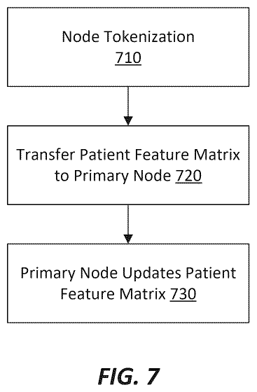

FIG. 7 is a flowchart that illustrates an example of adding a new node to a network of nodes. The new node can correspond to, for example, a new healthcare provider, a new pharmacy, a new data source within an existing organization, etc. At operation 710, the node can tokenize data located on or accessible by the node, for example as shown in FIG. 6. At operation 720, the node can transfer a patient feature matrix to a primary node. The patient feature matrix can indicate patients (e.g., token IDs) present on the node and an indication of whether or not each patient has data corresponding to particular features (e.g., particular diagnostic, procedural, or prescription codes). At operation 730, the primary node can update a patient feature matrix stored on or accessible by the primary node to include the information in the patient feature matrix transferred to the primary node at operation 720. The patient feature matrix can be used by the primary node when determining which remote nodes to send queries to for particular queries.

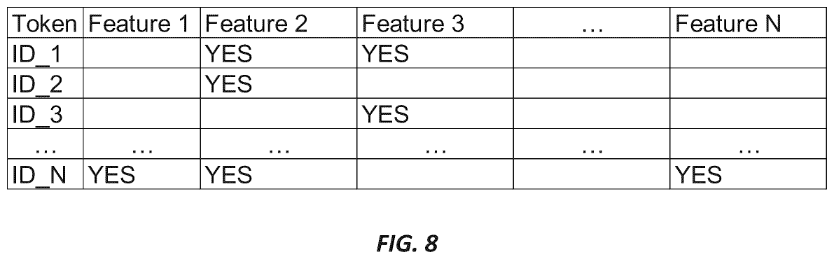

FIG. 8 is a drawing that illustrates an example patient feature matrix according to some implementations. The patient feature matrix can be used to easily determine which tokens (e.g., which patients) have certain features in their medical histories. The patient feature matrix can be used to determine which patients to include in a query or which nodes to send queries to. In some implementations, the patient feature matrix is stored on a primary node, and the primary node can use the patient feature matrix when generating queries to be executed on the primary node, one or more remote nodes, or both. As an example, if a query requires that Feature 2 be present in a patient health record, the primary node could use the patient feature matrix to determine that only records for patients ID_1, ID_2, and ID_N could possibly be relevant to the query, as the other patients shown in the patient feature matrix do not have values for Feature 2. In some implementations, the patient feature matrix indicates which nodes contain information for a patient. In some implementations, only nodes that have information for possibly relevant patients receive queries from the primary node.

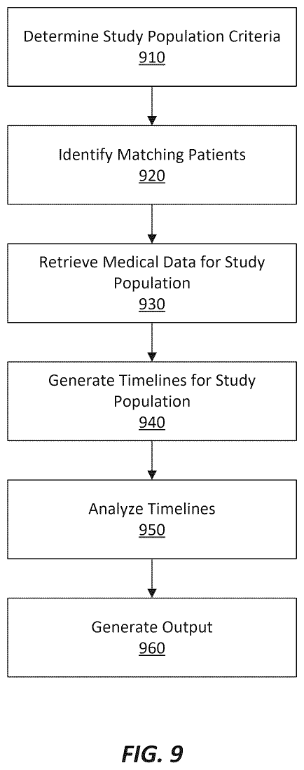

FIG. 9 is a flowchart that illustrates a process for retrieving and analyzing patient data according to some implementations. The process illustrated in FIG. 9 can be carried out on a computer system or multiple computer systems.

At operation 910, a system can determine study population criteria. For example, a user may submit a question or a study definition that includes criteria such as age, sex, gender, geographic area, socioeconomic status, medical conditions, and/or the like. At operation 920, the system can identify matching patients that meet the study population criteria, thereby defining the study population. In some implementations, the study population may be only partially defined. In some implementations, the study population at this stage includes all patients who could possibly match the study criteria, for example as determined from consulting a patient feature matrix. At operation 930, the system can retrieve medical data and/or additional data such as demographic data for the study population. At operation 940, the system can generate a patient timeline vector encoding for each patient included in the study population. At operation 950, the system can analyze the timelines for the patients in the study population. At operation 960, the system can generate an output. The output can summarize the analysis at operation 950. In some implementations, an output includes statistical summaries, written descriptions of results, links or citations to relevant research articles, or any combination thereof. Various operations illustrated in FIG. 9 can be carried out in different manners, which are described more fully herein, for example with reference to the subsequent drawings.

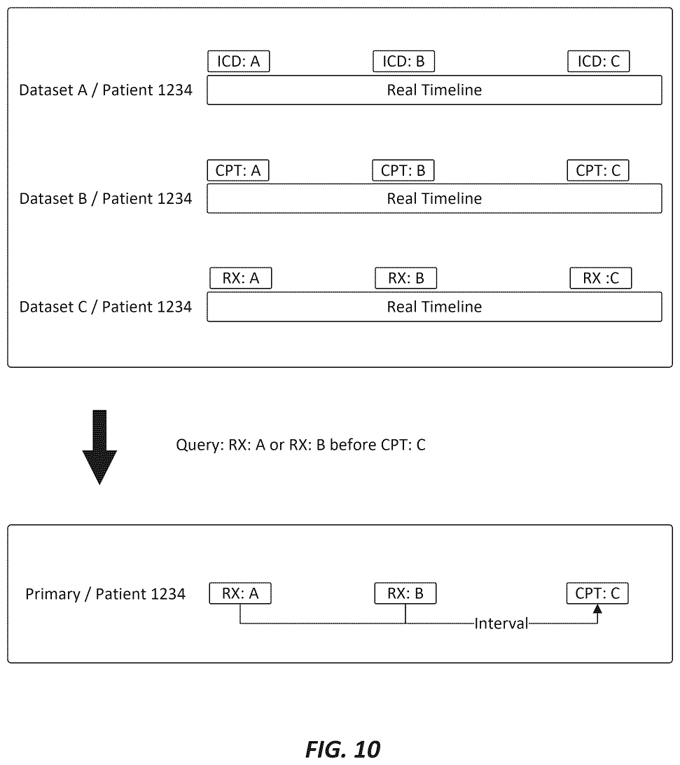

FIG. 10 is a diagram that schematically illustrates a patient timeline vector according to some implementations. In FIG. 10, information for a patient (Patient 1234) is distributed across three datasets (A, B, and C). In FIG. 10, Dataset A contains diagnostic information, Dataset B contains procedure information, and Dataset C contains medication information. However, it will be appreciated that such separation is not necessary. For example, each dataset can contain multiple types of data, for example, if a patient visits two different providers and has medical records in two different EHR systems, the information in the two EHR systems can include multiple types of information such as procedures, diagnoses, prescriptions, etc. A user can execute a query (e.g. “RX A or RX B before CPT C”) and the system can construct a patient timeline vector of the patient. The patient timeline vector can show, for example, that the patient was treated with RX A, then later treated with RX B, then finally had treatment C. The patient timeline vector can include interval information (e.g., the time between events on the patient timeline vector) but may not include specific dates.

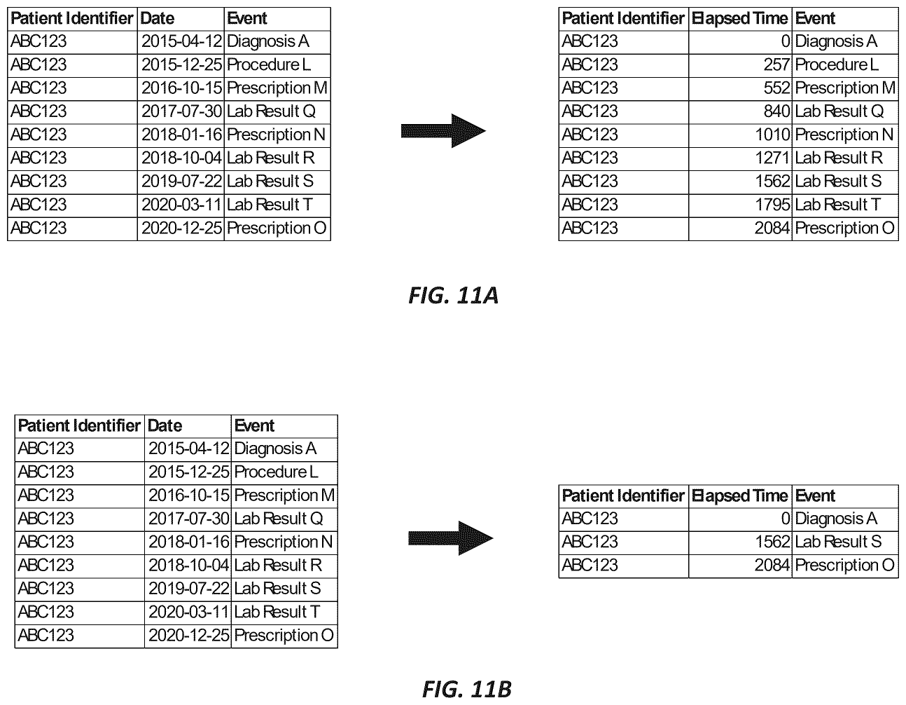

FIGS. 11A and 11B show examples of interval-encoded data according to some implementations. In FIG. 11A, a full record for a patient has been stripped of dates, and instead, the record contains time that elapsed since the patient received Diagnosis A. In FIG. 11B, the full record is not included, and instead, only certain information (e.g., information responsive to a query) is included. In FIGS. 11A and 11B, time is measured relative to Diagnosis A. However, time can be measured relative to any desired reference point, such as diagnosis date, treatment date, date a prescription was started or stopped, date of birth, date of death, the current date, a specific date in the past, etc.

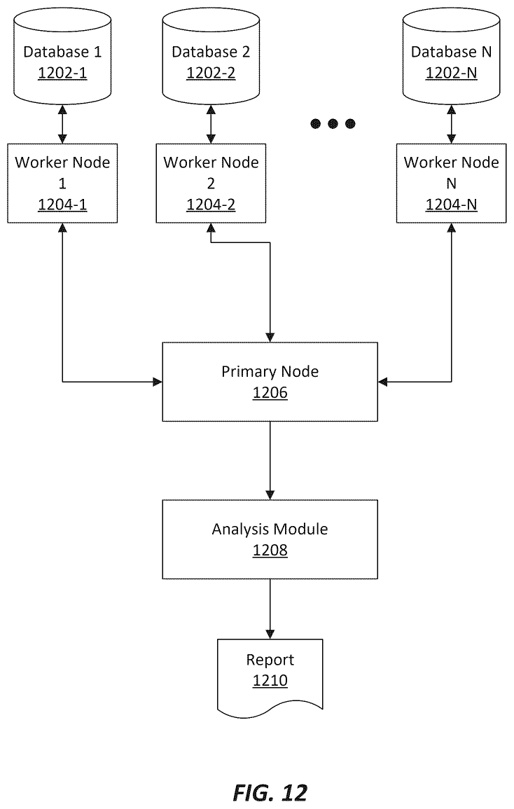

FIG. 12 is a block diagram that illustrates various components of a system according to the present disclosure. Some components, such as databases, can be part of the system or can be accessible by the system. A system can include a plurality of worker nodes 1204-1, 1204-N (collectively, worker nodes 1204 or individually, worker node 1204), a primary node 1206, and an analysis module 1208. The nodes can operate on different physical or virtual systems or can operate on the same physical or virtual system. Each of the worker nodes 1204 can have access to a database 1202-1, 1202-N (collectively, databases 1202 or individually, database 1202). In some implementations, a single worker node can have access to more than one database (e.g., a worker node for a hospital may have access to the hospital's EHR data and the hospital's pharmacy data). In some implementations, multiple worker nodes can have access to the same database.

Each worker node 1204 can be in communication with a corresponding database 1202. Each worker node 1204 can execute a query against its corresponding database 1202. The worker nodes 1204 can process information received from the databases 1202 to generate patient timeline sub-vectors. The patient timeline sub-vectors can include information responsive to the query. The worker nodes 1204 can calculate relative dates (intervals) measured against a specific reference date (e.g., the patient's date of birth or year of birth). The worker nodes 1204 can remove date information and include intervals in the patient timeline sub-vectors. In some implementations, all intervals can be measured relative to the same reference time. In some implementations, intervals can be measured relative to an adjacent event in a timeline. As an example, if a patient had office visits on March 1, April 1, and May 1, intervals could be determined as (assuming March 1 is taken as T=0) [0, 31, 61] or as [0, 31, 30]. In some implementations, intervals can be negative. For example, if May 1 is taken as T-0, intervals could be [−61, −31, 0].

The system can include a primary node 1206 that can receive the timeline sub-vectors and construct patient timeline vectors by combining the timeline sub-vectors. The patient timeline vectors can be provided to an analysis module 1208. The analysis module 1208 can carry out various analyses such as statistical calculations using the patient timeline vectors. The analysis module 1208 can be configured to output a report 1210. The report can summarize, explain, or otherwise provide aggregated information derived from the patient timeline vectors. The report 1210 may not include information about specific patients.

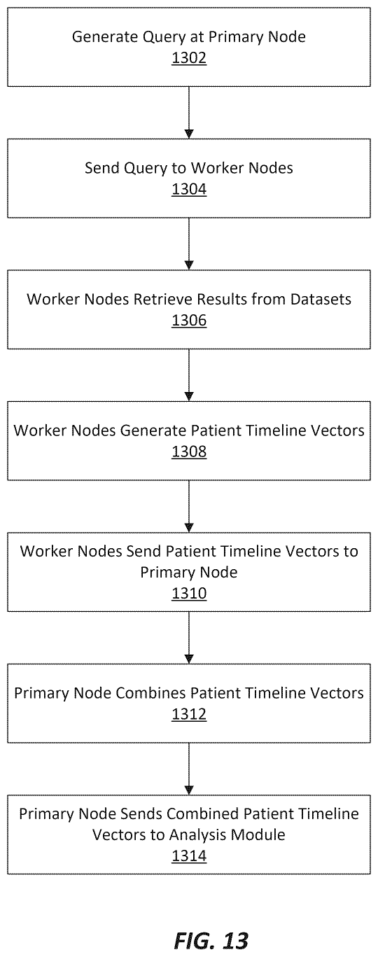

FIG. 13 illustrates an example process for generating and using patient timeline vectors according to some implementations. The process illustrated in FIG. 13 can be run on a system, such as the system illustrated in FIG. 12. At operation 1302, the system can generate a query at a primary node. At operation 1304, the primary node can send the query to one or more worker nodes for execution, for example as determined using a patient feature matrix. At operation 1306, the worker nodes can execute the query to retrieve results from corresponding datasets. At operation 1308, the worker nodes can generate patient timeline sub-vectors. At operation 1310, the worker nodes can send the patient timeline sub-vectors to the primary node. At operation 1312, the primary node can use the patient timeline sub-vectors to generate patient timeline vectors. At operation 1314, the primary node can send or otherwise make the patient timeline vectors available to an analysis module.

In some implementations, certain tasks are carried out by a primary node that is responsible for distributing queries to worker nodes, assembling patient timeline vectors from timeline sub-vectors, passing patient timeline vectors to an analysis module, and so forth. However, in some implementations, a primary node may not be used. Such an approach can reduce potential privacy concerns as there may be no node that is responsible for assembling complete patient timeline vectors.

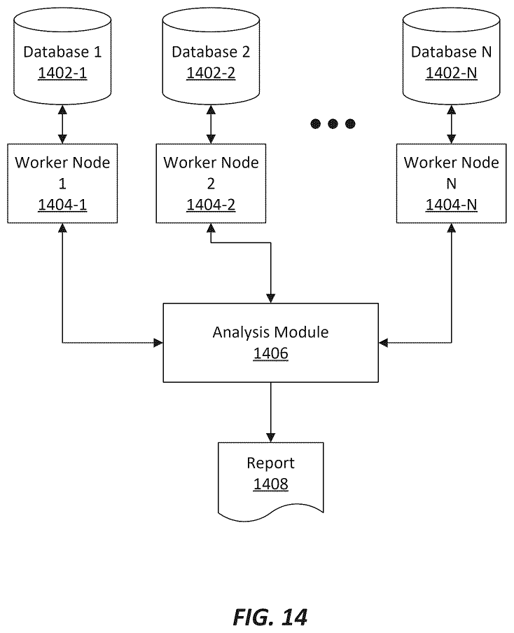

FIG. 14 is a block diagram that illustrates an example system and related features according to some implementations. The system depicted in FIG. 14 is generally similar to that depicted in FIG. 12, except that a primary node is not used.

Worker nodes 1404-1, 1404-N (collectively, worker nodes 1404 or individually, worker node 1404) can be in communication with databases 1402-1, 1402-N (collectively, databases 1402 or individually, database 1402). The worker nodes 1404 can execute a query against the databases 1402. Each worker node 1404 can construct a patient timeline sub-vector based on the results of the query. The worker nodes 1404 can pass the patient timeline sub-vectors to an analysis module 1406. The analysis module can combine the patient timeline sub-vectors and can perform analysis on the combined timeline vectors. The analysis module 1406 can generate a report 1408 that summarizes the results of the analysis or otherwise provides information related to the patient timeline sub-vectors. The report 1408 may not contain information about specific patients.

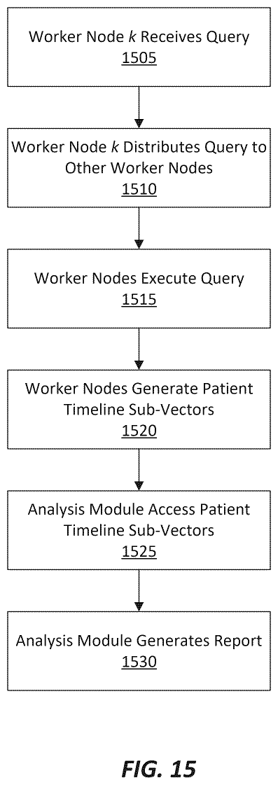

FIG. 15 is a flowchart that illustrates an example process for retrieving and analyzing patient data according to some implementations. At operation 1505, a worker node (worker node k) can receive a query. At operation 1510, worker node k can distribute the query to other worker nodes. At operation 1515, the worker nodes can execute the query against one or more datasets corresponding to each worker node. At operation 1520, the worker nodes can generate patient timeline sub-vectors. At operation 1525, an analysis module can access the generated patient timeline sub-vectors and can perform analysis on the sub-vectors. In some implementations, the analysis can include combining sub-vectors for particular patients to create patient timeline vectors. In some implementations, the created patient timeline vectors are not saved to non-volatile storage. At operation 1530, the analysis module can generate a report that summarizes results of the analysis.

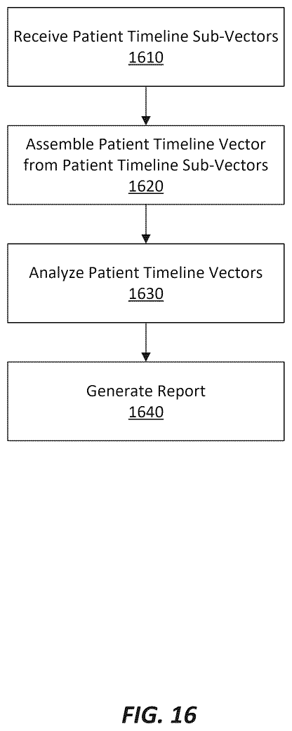

As described herein, in some implementations, it can be significant to combine sub-vectors to form patient timeline vectors, for example, to establish a more complete picture of a patient's history. FIG. 16 illustrates an example process for generating patient timeline vectors according to some implementations. At operation 1610, a system (e.g., a primary node) can receive patient timeline sub-vectors corresponding to a patient. At operation 1620, the system can assemble the patient timeline sub-vectors to create a patient timeline vector for the patient. At operation 1630, the system can analyze the patient timeline vector or can make the patient timeline vector available to another system for analysis. At operation 1640, the system, or the other system, can generate a report based on the analysis.

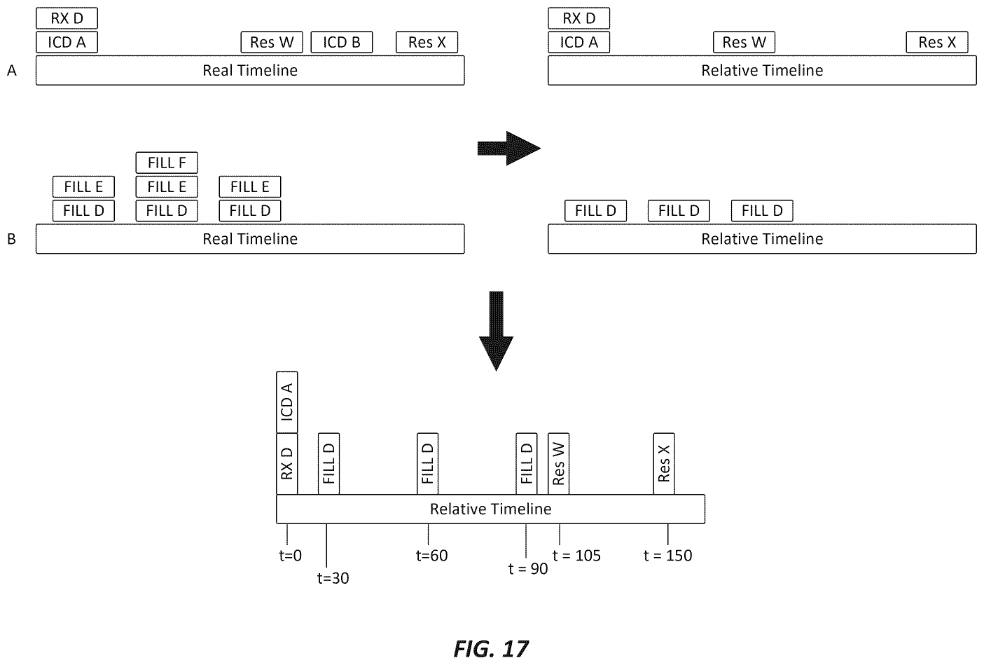

FIG. 17 schematically illustrates the generation of a patient timeline vector according to some implementations. In FIG. 17, information for a patient is contained in dataset A and dataset B. In FIG. 17, dataset B is a pharmacy dataset or pharmacy claims dataset that indicates which prescriptions were filled and when they were filled. However, it will be appreciated that in other cases, the datasets can be any combination of EHR data, pharmacy data, claims data, wearable health tracker data, etc.

In FIG. 17, dataset A contains information related to diagnoses, prescriptions, and lab results, while dataset B indicates when various prescriptions were filled. A researcher may not be interested in all of the information about the patient. Responsive to a query, a worker node can query Dataset A to retrieve relevant information (e.g., when the patient received diagnosis A, was prescribed medication D, received lab result W, and received lab result X) and place the relevant information on a relative timeline (in FIG. 17, measured relative to diagnosis A). The relative timeline can exclude irrelevant information or information not requested by the researcher, such as Diagnosis B. Dataset B indicates that the patient filled the prescription for medication D several times. Dataset B also indicates that the patient filled prescriptions E and F. In FIG. 17, prescriptions E and F are excluded from the relative timeline, which only indicates when prescription D was filled. The relative timelines can be combined to create a final patient timeline vector that indicates relevant events for the patient. In this way, the patient timeline vector can contain information that is of interest to the researcher while limiting the amount of information that is disclosed, thereby reducing the risk that a patient could be identified based on the patient timeline vector.

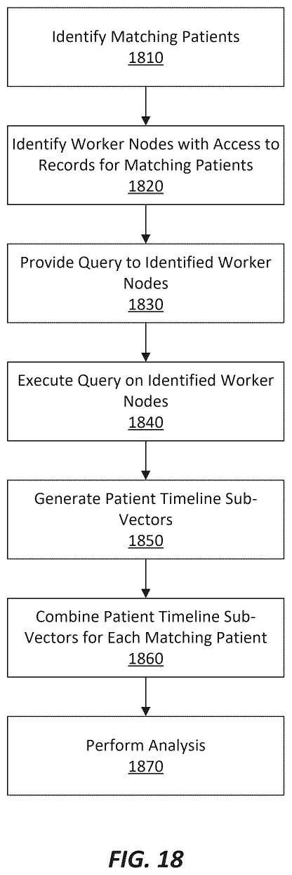

As described herein, it can be desirable to limit the number of queries that are performed in order to reduce costs, reduce server demands, improve performance, and so forth. Thus, in some implementations, queries may only be executed against datasets that contain information for patients in a study population. FIG. 18 is a flowchart that illustrates an example process for identifying datasets that contain data for patients in the study population and extracting information for those patients according to some implementations.

At operation 1810, a system can identify matching patients for a study population. In some implementations, matching patients can be potentially matching patients as determined using a patient feature matrix, and patients can be dropped if they ultimately do not meet the criteria to be included in the study population. In some implementations, the matching patients can be provided to the system or determined by the system as described herein. At operation 1820, the system can identify worker nodes with access to records for matching patients, for example, as described in more detail herein. At operation 1830, the system can provide a query to the worker nodes that have access to information for patients in the study population. At operation 1840, the worker nodes can execute queries against their corresponding datasets to retrieve information related to the patients in the study population. At operation 1850, the worker nodes can generate patient timeline sub-vectors, for example, as described herein. At operation 1860, the system can combine the patient timeline sub-vectors to generate patient timeline vectors for each patient in the study population. For example, a primary node or one of the worker nodes can combine the sub-vectors to generate patient timeline vectors. At operation 1870, the system can perform analysis on the patient timeline vectors.

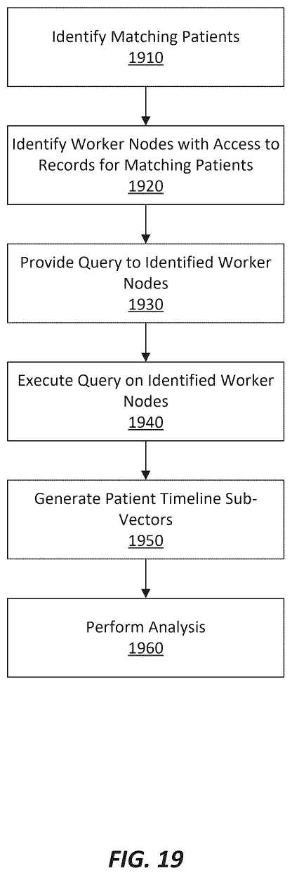

As described herein, in some implementations, the system may not generate complete patient timeline vectors. FIG. 19 is a flowchart that illustrates an example process for retrieving and processing patient information according to some implementations. The process shown in FIG. 19 is generally similar to that shown in FIG. 18. However, in FIG. 19, sub-vectors are not combined by a primary node or otherwise before being passed to an analysis module.

At operation 1910, a system can identify patients that match study criteria. At operation 1920, the system can identify worker nodes with access to records for the matching patients. At operation 1930, the worker nodes can access a query for retrieving information for the matching patients. At operation 1940, the worker nodes can execute the query against their corresponding datasets (e.g., against an in-memory cache of their corresponding datasets). At operation 1950, the worker nodes can generate patient timeline sub-vectors. At operation 1960, an analysis module can perform analysis using the patient timeline sub-vectors.

As described herein, in some implementations, patient data can be spread across multiple databases. That is, data for a single patient can be located in multiple datasets that are only accessible by different worker nodes. This can present a significant challenge for identifying patients that match study criteria, for example, when study population criteria are relatively complex and include multiple criteria.

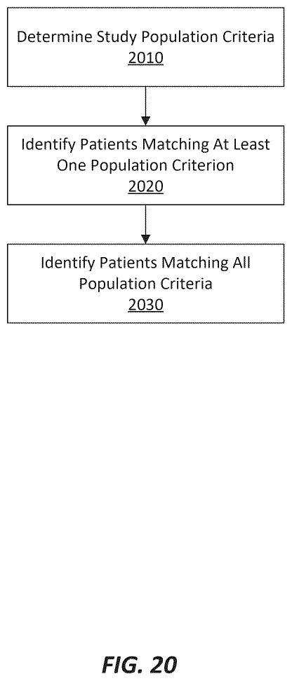

FIG. 20 is a flowchart that illustrates an example process for identifying patients who match population criteria according to some implementations. At operation 2010, a system can determine study population criteria, for example, by extracting information from a research proposal or question. In some implementations, a user can provide the study population criteria to the system, either directly or indirectly. At operation 2020, the system can identify patients who match one criterion or multiple criteria. For example, the system can execute queries on worker nodes that identify patients who match at least one criterion. As an example, if a population is specified as having the characteristics “Condition A and Condition B before Treatment C,” a primary node can identify patients in a patient feature matrix who have Condition A, Condition B, and Treatment C. At operation 2030, the system can find an intersection of patients who match all the criteria, thereby defining the study population. For example, once data is retrieved from the worker nodes, the primary node can determine if the patients in fact were diagnosed with conditions A and B before receiving treatment C.

The process shown in FIG. 20 can consume significant resources and may return large numbers of patients, many of whom do not satisfy all the criteria. In some implementations, a system can be configured to cascade searches. For example, a system can first search for patients who meet all the criteria within a single dataset. If a larger population is desired, the system may then look for patients who meet all the criteria but do not do so within a single dataset. In some implementations, a system can conduct queries in stages. For example, rather than searching for patients who match “Condition A or Condition B or Condition C,” a system may search for only one condition or a subset of conditions at a time. As an example, consider a study whose population criteria are “age >=65 AND diagnosis=small cell lung cancer.” While there are many patients who meet the age criteria, there are only about 20,000-30,000 new cases of small cell lung cancer per year in the United States. Thus, it can be advantageous to first identify patients with small cell lung cancer and then filter those patients to only those over 65. In some implementations, a system can access a database that includes disease prevalence data, and searches can be cascaded such that queries are conducted for patients with less prevalent diseases before searching for patients with more prevalent diseases.

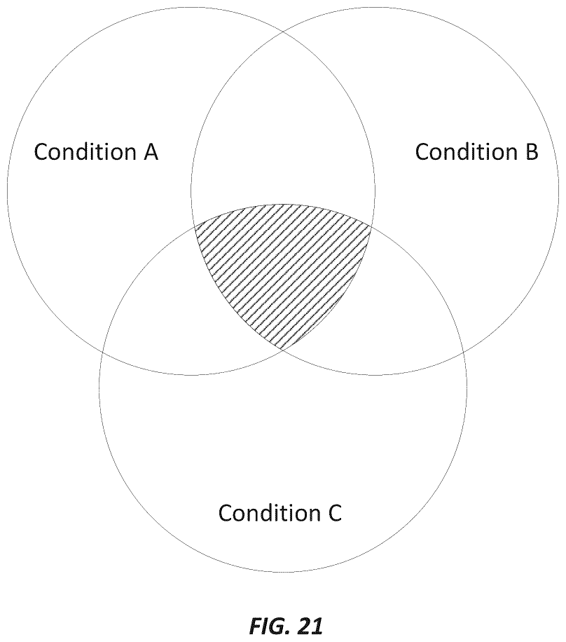

FIG. 21 is a drawing that illustrates a Venn diagram of patients matching one or more population criteria. In FIG. 21, three conditions are illustrated, although it will be appreciated that there can be more or fewer conditions. The shaded area indicates the subset of patients who match all three criteria, while the circles indicate all the patients matching Condition A, Condition B, or Condition C. It will be appreciated that the term “condition” is not strictly limited to medical conditions. For example, a “condition” can be a gender, sex, age, geographic location, prescription, treatment, medical diagnosis, etc.

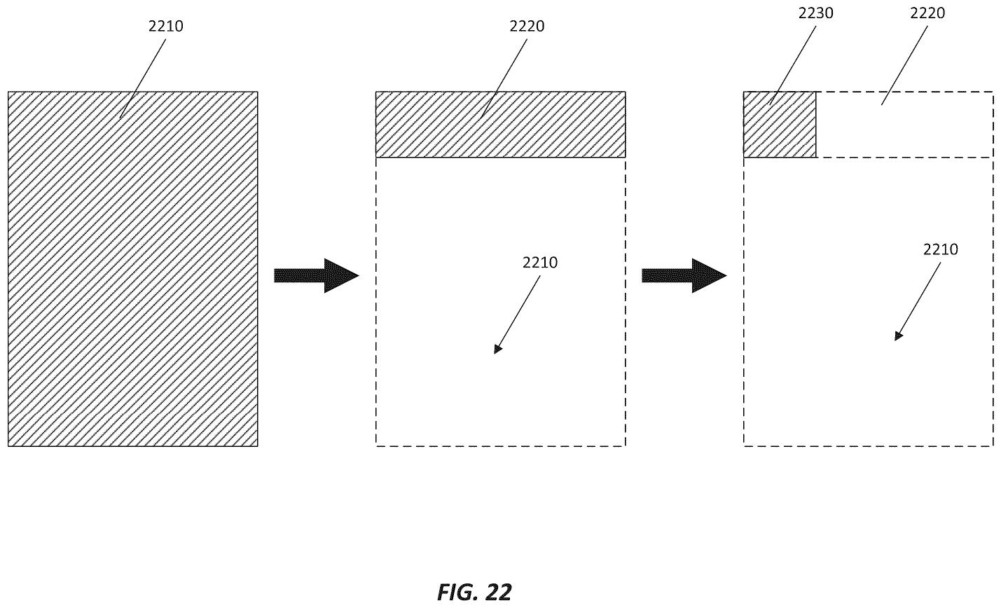

FIG. 22 illustrates an iterative approach to identifying patients matching study criteria according to some implementations. A first search for Condition A can return a population 2210. Condition A can be, in some implementations, the condition that is least likely to be true (e.g., a relatively uncommon disease, treatment, procedure, etc.). A subsequent query can be conducted only against patients in the population 2210 to refine the population to patients who satisfy Condition A and Condition B, giving a population 2220. A third search can be conducted for Condition C within the population 2220, giving a final population 2230 that satisfies Condition A, Condition B, and Condition C. As described herein, in some implementations, an approach can include identifying datasets that contain information about certain patients. Thus, in some implementations, one or more datasets can be excluded at each subsequent search. For example, if datasets A, B, C, D, E, and F are searched to identify the population 2210, and population 2210 is found within datasets A, B, C, and D, then datasets E and F can be excluded from the next search. If the population 2220 is found only within datasets B and D, then only datasets B and D may be used in the third search. Thus, the total number of queries to be executed can, in some cases, be reduced by narrowing down which datasets need to be searched to identify patients at each subsequent refinement step.

As described herein, data for a single patient can be spread across multiple datasets that are accessible by different worker nodes. It can be significant to reduce the number of datasets that are queried when searching for patient information. In some implementations, each worker node or the primary node can access a dataset that indicates where information for specific patients can be found (e.g., which worker nodes to use to access information for a specific patient and/or which datasets to access to retrieve information for a specific patient). In some implementations, it may be undesirable or infeasible for each worker node to maintain a complete record of patients and where their information can be found. For example, there may be a large number of patients, such as millions, tens of millions, or more. Moreover, there may be increased data security or privacy concerns if a single worker node or other system has a complete record of all the datasets where information for a particular patient is located. Thus, as described herein, it can be significant to distribute such information across multiple worker nodes or other systems.

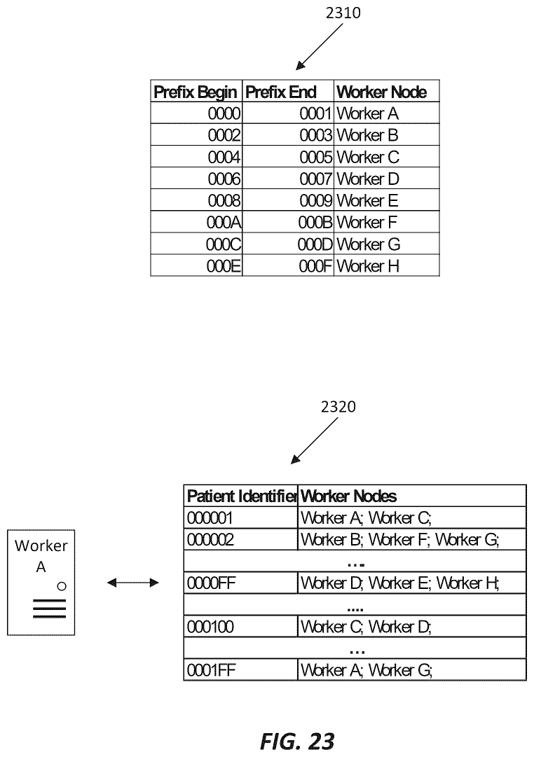

FIG. 23 schematically illustrates routing tables that can be used to locate patient records according to some implementations. In FIG. 23, the index table 2310 indicates which systems (e.g., worker nodes) contain information about where patient data is located. Routing table 2320 indicates the specific nodes or locations where patient information is located. In some implementations, each worker node can have a copy of or can access the index table 2310. In some implementations, a primary node can store or access the index table 2310. When a patient identifier is received by a node, the node can look up a patient identifier or a portion thereof in the index table 2310. For example, in the index table 2310, only patient identifier prefixes are illustrated (e.g., the first four (padded) digits of a hexadecimal-encoded identifier). The index table indicates which worker node has routing information for patients beginning with certain prefixes. After a worker node is identified from the index table 2310, the relevant node can execute a query to determine which nodes have access to the patient information. For example, in FIG. 23, the index table 2310 indicates that patient identifiers beginning with ‘0000’ through ‘0001’ can be looked up on Worker A. Worker A has or can access the routing table 2320, which indicates which nodes contain information for those patients. As an example, consider a lookup for a patient whose identifier is ‘0000FF.’ A query of the index table 2310 indicates that routing information for ‘0000FF’ is located on Worker A. The routing table 2320 on Worker A indicates that records for patient identifier ‘0000FF’ are located on Worker D, Worker E, and Worker H (e.g., that Worker D, Worker E, and Worker H have access to data for patient ‘0000FF’). Using this information, the number of queries to retrieve information for patient ‘0000FF’ can be reduced, as instead of querying for patient records on Workers A-H, only three workers (D, E, and H) may be queried, as they are the only workers that contain information for patient ‘0000FF.’

It will be appreciated that other approaches are possible. For example, instead of indexing based on prefixes, indexes can be based on entire patient identifiers, suffixes, etc. In some implementations, for example, when there is a limited number of patients such that there is little or no benefit to dividing the routing table across systems, each system can access or have a copy of a routing table, which can be used to determine whether or not to execute a query for a particular patient. For example, consider a scenario in which each worker node receives a query. The worker nodes can query the routing tables 2320 to determine if they should execute the query. For example, if at least one patient specified in the query has patient records that are accessible by the node, the node can execute the query, but may not execute the query if the node does not have access to patient records for any of the patients specified in the query. In some implementations, a node can modify a query. For example, if Worker A receives a query that includes patients [000001, 0000FF, 0001FF], worker A can modify the query that it executes to only include patients [000001, 0001FF], as the routing table 2320 indicates that Worker A does not have access to patient records for patient 0000FF. In some implementations, a worker node does not modify a query.

Another difficulty associated with decentralized patient data is determining a reference date for interval encodings. In some cases, the reference date can be straightforward. For example, a researcher might use the current date or a specific date in the past as the reference date. In some implementations, however, determining the reference date can be more complex. For example, a reference date can be based on a diagnosis date, a procedure date, a date when a treatment was started, a patient's date of birth, a patient's date of death, etc. The relevant information for determining the reference date may not be present in every dataset. For example, an EHR dataset may include a diagnosis date for a patient, but such information is unlikely to be present in a pharmacy claims dataset. Thus, a worker node that retrieves information from the pharmacy claims dataset may not itself be able to determine the appropriate reference date for calculating intervals.

In some implementations, worker nodes without the reference date can use a default date or a date supplied as part of a request to execute a query. In some implementations, the approaches herein can be used to provide the reference date to worker nodes, for example, in the form of a vector or data frame that includes a patient identifier and a reference date, such as [ABC1234, 2019 Mar. 12].

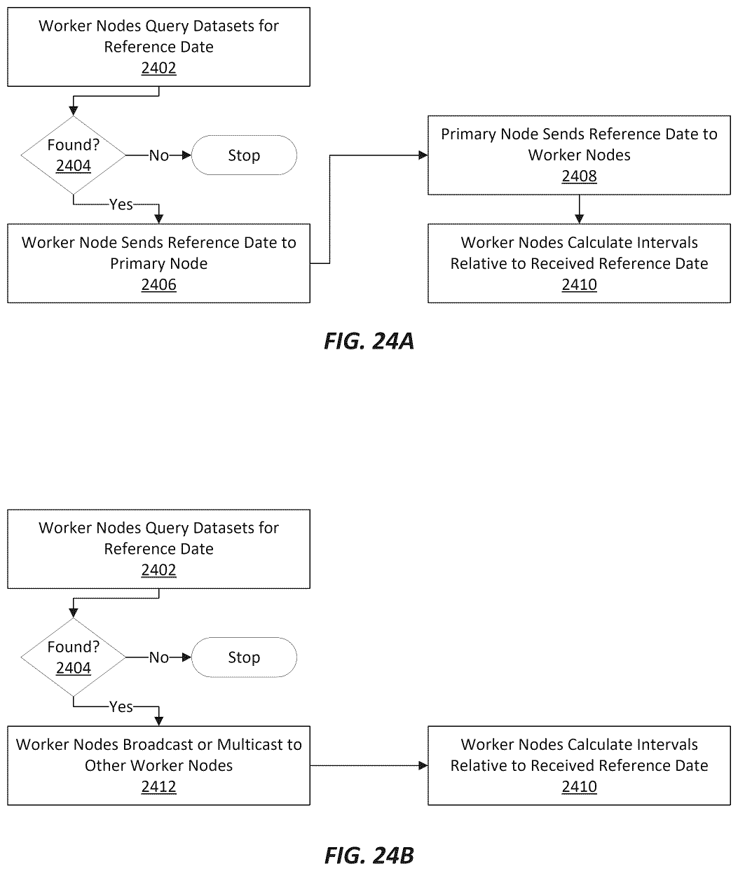

FIG. 24A illustrates an example process for sharing a reference date among worker nodes according to some implementations. At operation 2402, worker nodes can query their corresponding datasets to determine if they have access to the reference date. At operation 2404, each worker node can determine if it has determined the reference date or not. If not, the worker node can stop. If so, the worker node can provide the reference date to a primary node at operation 2406. At operation 2408, the primary node can provide the reference date to the rest of the worker nodes. At operation 2410, the worker nodes can calculate intervals using the received reference date.

In FIG. 24A, a primary node is used to distribute the reference date to the worker nodes. However, this may implicate patient privacy, as the primary node also receives the patient timeline sub-vectors and assembles a patient timeline vector in some implementations, meaning that the primary node can have access to both intervals and an absolute reference date.

FIG. 24B illustrates a decentralized approach to sharing a reference date among worker nodes according to some implementations. At operation 2402, worker nodes can query their corresponding datasets to determine if they have access to the reference date. At operation 2404, each worker node can determine if it has determined the reference date or not. If not, the worker node can stop. If so, the worker node can broadcast or multicast the reference date to the other worker nodes at operation 2412. For example, the worker node can broadcast the reference date to all other worker nodes or can multicast the reference date to nodes that contain relevant patient information to be retrieved, if the worker node has knowledge of which nodes contain relevant patient information, for example, as described herein. At operation 2410, the worker nodes can calculate intervals using the received reference date.

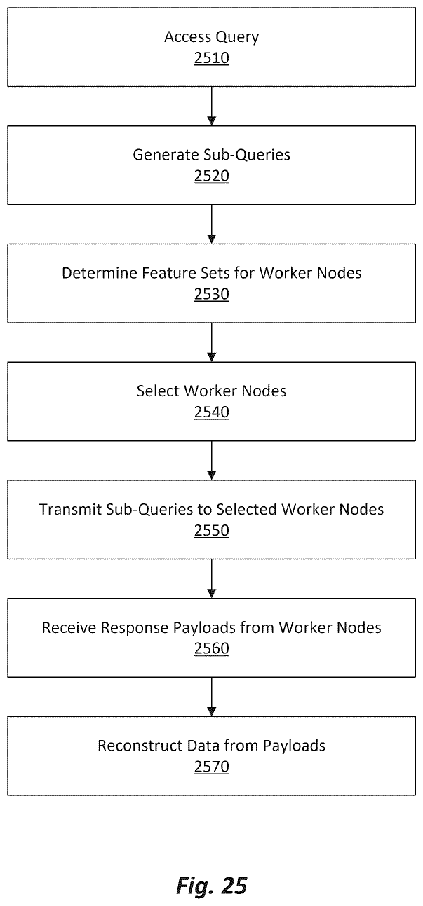

FIG. 25 is a flowchart that illustrates an example data retrieval process according to some implementations. The process shown in FIG. 25 can be used, for example, when retrieving larger portions of data from worker nodes, as opposed to simple cases where simple timeline vectors are sufficient. For example, the process shown in FIG. 25 can be used when transmitting raw patient data in compressed form.

At operation 2510, a primary node can access a query, for example, a query submitted by a user or constructing by another software program. At operation 2520, the primary node can generate one or more sub-queries as described herein. At operation 2530, the primary node can access feature set information for one or more worker nodes, which can indicate which patients or worker nodes could possibly have data relevant to the original query. At operation 2540, the system can select worker nodes, patients, or both, based on whether or not they potentially have relevant data. At operation 2550, the primary node can transmit sub-queries to selected worker nodes. The worker nodes can respond by transmitting raw data to the primary node. The raw data can be in compressed (e.g., binary compressed) form. In some implementations, the raw data can be encrypted. In some implementations, data other than raw data can be transmitted. Further, it will be appreciated that raw data may undergo one or more manipulations, such as removing PII or substituting relative dates for actual dates.

At operation 2560, the primary node can receive response payloads from the worker nodes. The primary node can reconstruct data from the payloads at operation 2570 to enable further processing and analysis. The payloads can include timelines, binary compressed tables, or any other relevant data.

### Example Clauses

Embodiment 1. A computer-implemented method for deidentified data processing, the computer-implemented method comprising: accessing, by a primary node, a query, wherein the query includes a plurality of population conditions for defining a cohort to be included in a retrospective medical study; accessing a plurality of patient feature set matrices, wherein each patient feature set matrix of the plurality of patient feature set matrices corresponds to a worker node of the plurality of worker nodes, wherein each patient feature set matrix indicates data present on or accessible by the corresponding worker node, wherein each patient feature set matrix indicates which patients have data corresponding to a plurality of features; determining, based on the plurality of population conditions and the plurality of patient feature set matrices, a set of target worker nodes and a set of patients, wherein the target worker nodes of the set of target worker nodes are selected from the plurality of worker nodes, wherein each target worker node of the set of target worker nodes is a node that is determined to possibly have data responsive to the query, wherein the set of patients is selected by identifying patients with data corresponding to a population condition; causing execution of the worker node queries on the identified set of target worker nodes; accessing results returned by the target worker nodes of the set of target worker nodes; generating a dataset comprising patient timeline objects using the results, wherein the patient timeline objects indicate a presence of a feature at a time, wherein the time is measured relative to a reference date; and making the dataset available for use.

Embodiment 2. The computer-implemented method of embodiment 1, wherein the results returned by the target worker nodes comprise binary compressed data, and wherein generating the dataset comprising patient timeline objects comprising reconstructing the results from the binary compressed data to a table format.

Embodiment 3. The computer-implemented method of embodiment 1, further comprising purging the dataset from a volatile memory.

Embodiment 4. A computer-implemented method for deidentified data processing, the computer-implemented method comprising: accessing, by a primary node, a query, wherein the query includes a plurality of population conditions for defining a cohort for a retrospective medical study; determining, by the primary node using one or more feature set matrices, a first set of patients, wherein the first set of patients includes patients who could be included in the cohort based on one or more population conditions of the plurality of population conditions; generating, by the primary node, one or more requests for data from one or more worker nodes, wherein the one or more requests are configured to request data associated with the first set of patients from the one or more worker nodes; transmitting, by the primary node, the one or more requests to the one or more worker nodes; accessing, by the primary node, a plurality of responses from a plurality of worker nodes, the responses generated by the worker nodes in response to executing the one or more generated queries; generating, by the primary node, a dataset based on the plurality of responses, wherein the dataset comprises a plurality of patient timeline vectors for a plurality of cohort patients, wherein at least one of the plurality of patient timeline vectors is generated by combining a first response from a first worker node and a second response from a second worker node that is different from the first worker node.

Embodiment 5. The computer-implemented method of embodiment 4, wherein the worker node performs a safe harbor check prior to making the data available to the primary node.

Embodiment 6. The computer-implemented method of embodiment 4, wherein patients are identified across worker nodes by tokens, wherein the tokens are generated using a combination of two or more of: patient first name, patient last name, patient date of birth, patient social security number, or patient address.

Embodiment 7. The computer-implemented method of embodiment 4, wherein the primary node stores the dataset in volatile memory, wherein the primary node deletes the dataset from the volatile memory based on at least one of a completion of a data analysis process or an expiration of a time to live.

Embodiment 8. The computer-implemented method of embodiment 4, wherein the data includes one or more of diagnosis data, clinical notes, imaging data, laboratory results, prescription data, or pharmacy claims data.

Embodiment 9. The computer-implemented method of embodiment 4, further comprising, prior to executing the requests, determining a rank ordering of the requests, wherein the rank ordering is determined using relative frequencies of conditions included in the requests, wherein less commonly occurring conditions are ranked higher than more commonly occurring conditions, wherein requests are executed in rank order.

Embodiment 10. The computer-implemented method of embodiment 9, wherein a second, subsequent request is constrained based on a result of a first, earlier request.

Embodiment 11. The computer-implemented method of embodiment 4, further comprising: analyzing the patient data based on a specified analysis; and generating a report of the analysis.

Embodiment 12. The computer-implemented method of embodiment 4, wherein the responses comprise a plurality of patient timeline sub-vectors, wherein the patient timeline sub-vectors indicate events relative to a reference date.

Embodiment 13. The computer-implemented method of embodiment 12, wherein the reference date for a patient timeline sub-vector is the date of birth for a patient associated with the patient timeline sub-vector.

Embodiment 14. The computer-implemented method of embodiment 4, wherein the responses comprise compressed data table representations of raw patient data, wherein generating the dataset comprises reconstructing a plurality of data tables from the compressed data table representations.

Embodiment 15. The computer-implemented method of embodiment 14, wherein the reconstructed data tables represent dates as relative dates from a reference date, wherein the reference date is a date of birth of a patient associated with a reconstructed data table, wherein the reconstructed data table is associated with a single patient.

Embodiment 16. A system comprising: one or more hardware processors; and a non-transitory computer-readable storage medium having instructions stored thereon that, when executed by the one or more hardware processors, cause the system to: generate a query, wherein the query includes a plurality of population conditions for defining a cohort for a retrospective medical study; determine, using one or more feature set matrices, a first set of patients, wherein the first set of patients includes patients who could be included in the cohort based on one or more population conditions of the plurality of population conditions; generate one or more requests for data from one or more worker nodes, wherein the one or more requests are configured to request data associated with the first set of patients from the one or more worker nodes; transmit the one or more requests to the one or more worker nodes; access a plurality of responses from a plurality of worker nodes, the responses generated by the worker nodes in response to executing the one or more generated queries; generate a dataset based on the plurality of responses, wherein the dataset comprises a plurality of patient timeline vectors for a plurality of cohort patients, wherein at least one of the plurality of patient timeline vectors is generated by combining a first response from a first worker node and a second response from a second worker node that is different from the first worker node.

Embodiment 17. The system of embodiment 16, wherein the system stores the data from the worker node in volatile memory, wherein the system deletes the data from the volatile memory based on at least one of a completion of a data analysis process or an expiration of a time to live.

Embodiment 18. The system of embodiment 16, wherein the responses comprise a plurality of patient timeline sub-vectors, wherein the patient timeline sub-vectors indicate events relative to a reference date.

Embodiment 19. The system of embodiment 16, wherein the responses a plurality of compressed data table representations, wherein the instructions are further configured to cause the system to reconstruct a plurality of data tables from the compressed data table representations.

Embodiment 20. The system of embodiment 19, wherein the reconstructed data tables represent dates as relative dates from a reference date, wherein the reference data is a date of birth of a patient associated with a reconstructed data table, wherein the reconstructed data table is associated with a single patient.

### Computer System

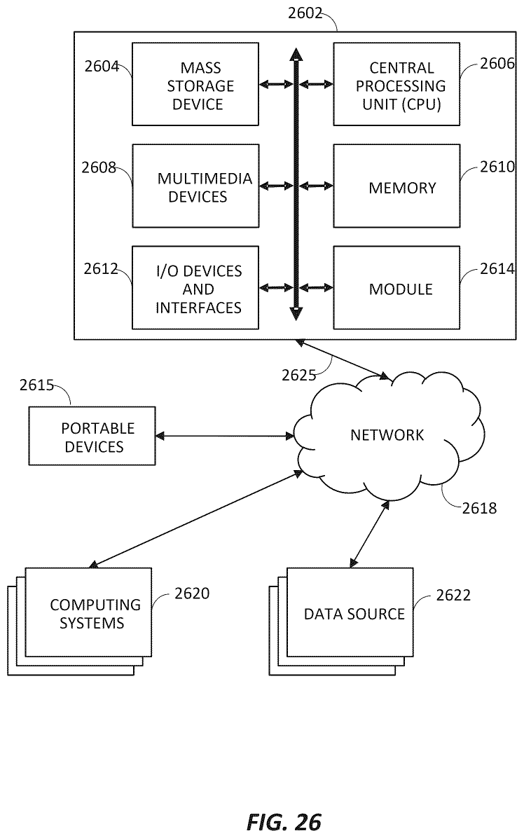

FIG. 26 is a block diagram 2600 depicting an embodiment of a computer hardware system 2602 configured to run software for implementing one or more of the systems and methods described herein. The computer system shown in FIG. 26 is merely an example, and it will be appreciated that the systems and methods herein can be run on other suitable computing systems, which may have fewer, more, and/or different features than the example shown in FIG. 26. Moreover, the approaches described herein are not limited to being performed by a single computer system, unless context clearly indicates otherwise.

The example computer system 2602 is in communication with one or more computing systems 2620, portable devices 2615, and/or one or more data sources 2622 via one or more networks 2618. While FIG. 26 illustrates an embodiment of a computing system 2602, it is recognized that the functionality provided for in the components and modules of computer system 2602 may be combined into fewer components and modules, or further separated into additional components and modules.

The computer system 2602 can comprise a module 2614 that carries out the functions, methods, acts, and/or processes described herein. The module 2614 is executed on the computer system 2602 by a central processing unit 2606 discussed further below.

In general, the word “module,” as used herein, refers to logic embodied in hardware or firmware or to a collection of software instructions, having entry and exit points. Modules are written in a programming language, such as Java, C or C++, Python, or the like. Software modules may be compiled or linked into an executable program, installed in a dynamic link library, or may be written in an interpreted language such as BASIC, PERL, Lua, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some implementations, process blocks described herein may be altered, rearranged, combined, and/or omitted.

The computer system 2602 includes one or more processing units (CPU) 2606, which may comprise a microprocessor. The computer system 2602 further includes a physical memory 2610, such as random-access memory (RAM) for temporary storage of information, a read only memory (ROM) for permanent storage of information, and a mass storage device 2604, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 2602 are connected to the computer using a standards-based bus system. The bus system can be implemented using various protocols, such as Peripheral Component Interconnect (PCI), PCI Express, Micro Channel, SCSI, Industrial Standard Architecture (ISA) and Extended ISA (EISA) architectures. In some implementations, for example when using a cloud service such as Amazon Web Services, there may not be a mass storage device attached directly to the computer system. For example, storage operations can be made against a remote datastore via a network connection. In some cases, there may be some amount of local storage, e.g., to facilitate temporary storage.

The computer system 2602 includes one or more input/output (I/O) devices and interfaces 2612, such as a keyboard, mouse, touch pad, and printer. The I/O devices and interfaces 2612 can include one or more display devices, such as a monitor, which allows the visual presentation of data to a user. More particularly, a display device provides for the presentation of GUIs as application software data, and multi-media presentations, for example. The I/O devices and interfaces 2612 can also provide a communications interface to various external devices. The computer system 2602 may comprise one or more multi-media devices 2608, such as speakers, video cards, graphics accelerators, and microphones, for example. In some implementations, a computer system may not have certain devices, such as certain I/O devices, a display, etc.

The computer system 2602 may run on a variety of computing devices, such as a server, a Windows server, a Structure Query Language server, a Unix Server, a personal computer, a laptop computer, and so forth. In other implementations, the computer system 2602 may run on a cluster computer system, a mainframe computer system and/or other computing system suitable for controlling and/or communicating with large databases, performing high volume transaction processing, and generating reports from large databases. The computing system 2602 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide filesystem, networking, and I/O services, and provide a user interface, such as a graphical user interface (GUI), among other things.

The computer system 2602 illustrated in FIG. 26 is coupled to a network 2618, such as a LAN, WAN, or the Internet via a communication link 2616 (wired, wireless, or a combination thereof). Network 2618 communicates with various computing devices and/or other electronic devices, such as portable devices 2615. Network 2618 is communicating with one or more computing systems 2620 and one or more data sources 2622. The module 2614 may access or may be accessed by computing systems 2620 and/or data sources 2622 through a web-enabled user access point. Connections may be a direct physical connection, a virtual connection, and other connection type. The web-enabled user access point may comprise a browser module that uses text, graphics, audio, video, and other media to present data and to allow interaction with data via the network 2618.

Access to the module 2614 of the computer system 2602 by computing systems 2620 and/or by data sources 2622 may be through a web-enabled user access point such as the computing systems' 2620 or data source's 2622 personal computer, cellular phone, smartphone, laptop, tablet computer, e-reader device, audio player, or another device capable of connecting to the network 2618. Such a device may have a browser module that is implemented as a module that uses text, graphics, audio, video, and other media to present data and to allow interaction with data via the network 2618.

The output module may be implemented as a combination of an all-points addressable display such as a cathode ray tube (CRT), a liquid crystal display (LCD), a plasma display, or other types and/or combinations of displays. The output module may be implemented to communicate with interfaces 2612 and they also include software with the appropriate interfaces, which allow a user to access data through the use of stylized screen elements, such as menus, windows, dialogue boxes, toolbars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some implementations, the system 2602 may comprise a physical or logical connection established between a remote microprocessor and a mainframe host computer for the express purpose of uploading, downloading, or viewing interactive data and databases on-line in real time. The remote microprocessor may be operated by an entity operating the computer system 2602, including the client server systems or the main server system, an/or may be operated by one or more of the data sources 2622 and/or one or more of the computing systems 2620. In some implementations, terminal emulation software may be used on the microprocessor for participating in the micro-mainframe link.

In some implementations, computing systems 2620 who are internal to an entity operating the computer system 2602 may access the module 2614 internally as an application or process run by the CPU 2606.

In some implementations, one or more features of the systems, methods, and devices described herein can utilize a URL and/or cookies, for example, for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, a domain name, a file extension, a host name, a query, a fragment, a scheme, a protocol identifier, a port number, a username, a password, a flag, an object, a resource name, and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a website). The cookie data can be encrypted to provide security for the creator. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 2602 may include one or more internal and/or external data sources (for example, data sources 2622). In some implementations, one or more of the data repositories and the data sources described above may be implemented using a relational database, such as DB2, Sybase, Oracle, CodeBase, and Microsoft® SQL Server, as well as other types of databases, such as a flat-file database, an entity relationship database, an object-oriented database, and/or a record-based database.

The computer system 2602 may also access one or more databases 2622. The databases 2622 may be stored in a database or data repository. The computer system 2602 may access the one or more databases 2622 through a network 2618 or may directly access the database or data repository through I/O devices and interfaces 2612. The data repository storing the one or more databases 2622 may reside within the computer system 2602.

Unless context indicates otherwise, methods described herein can be run on a single computer system or multiple computer systems. Computer systems can include physical computer systems, virtual computer systems (e.g., virtual machines), or both. The methods herein can, in some implementations, be carried out in containers, which can act as a silo for running an application or applications on a host operating system. In some implementations, a computer system can be headless and may not include a display device. In some implementations, a computer system may not include or be connected to an input device such as a mouse or keyboard.

### CONCLUSION

In the foregoing specification, the systems and processes have been described with reference to specific implementations thereof. It will, however, be evident that various modifications and changes may be made thereto without departing from the broader spirit and scope of the implementations disclosed herein. The specification and drawings are, accordingly, to be regarded in an illustrative rather than restrictive sense.

Indeed, although the systems and processes have been disclosed in the context of certain implementations and examples, it will be understood by those skilled in the art that the various implementations of the systems and processes extend beyond the specifically disclosed implementations to other alternative implementations and/or uses of the systems and processes and obvious modifications and equivalents thereof. In addition, while several variations of the implementations of the systems and processes have been shown and described in detail, other modifications, which are within the scope of this disclosure, will be readily apparent to those of skill in the art based upon this disclosure. It is also contemplated that various combinations or sub-combinations of the specific features and aspects of the implementations may be made and still fall within the scope of the disclosure. It should be understood that various features and aspects of the disclosed implementations can be combined with, or substituted for, one another in order to form varying modes of the implementations of the disclosed systems and processes. Any methods disclosed herein need not be performed in the order recited. Thus, it is intended that the scope of the systems and processes herein disclosed should not be limited by the particular implementations described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate implementations also may be implemented in combination in a single embodiment. Conversely, various features that are described in the context of a single embodiment also may be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain implementations include, while other implementations do not include, certain features, elements and/or operations. Thus, such conditional language is not generally intended to imply that features, elements and/or operations are in any way required for one or more implementations or that one or more implementations necessarily include logic for deciding, with or without author input or prompting, whether these features, elements and/or operations are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other implementations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the implementations are not to be limited to the particular forms or methods disclosed, but, to the contrary, the implementations are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or implementations set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example, ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain implementations require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the implementations shown herein but are to be accorded the widest scope consistent with this disclosure, the principles and the novel features disclosed herein.

What is claimed is:

1. A computer-implemented method for deidentified data processing, the computer-implemented method comprising:
accessing, by a primary node, a query,
wherein the query includes a plurality of population conditions for defining a cohort to be included in a retrospective medical study;
accessing a plurality of patient feature set matrices,
wherein each patient feature set matrix of the plurality of patient feature set matrices corresponds to a worker node of the plurality of worker nodes,
wherein each patient feature set matrix indicates data present on or accessible by the corresponding worker node,
wherein each patient feature set matrix indicates which patients have data corresponding to a plurality of features;
determining, based on the plurality of population conditions and the plurality of patient feature set matrices, a set of target worker nodes and a set of patients,
wherein the target worker nodes of the set of target worker nodes are selected from the plurality of worker nodes,
wherein each target worker node of the set of target worker nodes is a node that is determined to possibly have data responsive to the query,
wherein the set of patients is selected by identifying patients with data corresponding to a population condition;
causing execution of the worker node queries on the identified set of target worker nodes;
accessing results returned by the target worker nodes of the set of target worker nodes;
generating a dataset comprising patient timeline objects using the results, wherein the patient timeline objects indicate a presence of a feature at a time, wherein the time is measured relative to a reference date; and
making the dataset available for use.

2. The computer-implemented method of claim 1, wherein the results returned by the target worker nodes comprise binary compressed data, and wherein generating the dataset comprising patient timeline objects comprising reconstructing the results from the binary compressed data to a table format.

3. The computer-implemented method of claim 1, further comprising purging the dataset from a volatile memory.

4. A computer-implemented method for deidentified data processing, the computer-implemented method comprising:
accessing, by a primary node, a query, wherein the query includes a plurality of population conditions for defining a cohort for a retrospective medical study;
determining, by the primary node using one or more feature set matrices, a first set of patients, wherein the first set of patients includes patients who could be included in the cohort based on one or more population conditions of the plurality of population conditions;
generating, by the primary node, one or more requests for data from one or more worker nodes, wherein the one or more requests are configured to request data associated with the first set of patients from the one or more worker nodes;
transmitting, by the primary node, the one or more requests to the one or more worker nodes;
accessing, by the primary node, a plurality of responses from a plurality of worker nodes, the responses generated by the worker nodes in response to executing the one or more generated queries;
generating, by the primary node, a dataset based on the plurality of responses, wherein the dataset comprises a plurality of patient timeline vectors for a plurality of cohort patients, wherein at least one of the plurality of patient timeline vectors is generated by combining a first response from a first worker node and a second response from a second worker node that is different from the first worker node.

5. The computer-implemented method of claim 4, wherein the worker node performs a safe harbor check prior to making the data available to the primary node.

6. The computer-implemented method of claim 4, wherein patients are identified across worker nodes by tokens, wherein the tokens are generated using a combination of two or more of: patient first name, patient last name, patient date of birth, patient social security number, or patient address.

7. The computer-implemented method of claim 4, wherein the primary node stores the dataset in volatile memory, wherein the primary node deletes the dataset from the volatile memory based on at least one of a completion of a data analysis process or an expiration of a time to live.

8. The computer-implemented method of claim 4, wherein the data includes one or more of diagnosis data, clinical notes, imaging data, laboratory results, prescription data, or pharmacy claims data.

9. The computer-implemented method of claim 4, further comprising, prior to executing the requests, determining a rank ordering of the requests,
wherein the rank ordering is determined using relative frequencies of conditions included in the requests,
wherein less commonly occurring conditions are ranked higher than more commonly occurring conditions,
wherein requests are executed in rank order.

10. The computer-implemented method of claim 9, wherein a second, subsequent request is constrained based on a result of a first, earlier request.

11. The computer-implemented method of claim 4, further comprising:
analyzing the patient data based on a specified analysis; and
generating a report of the analysis.

12. The computer-implemented method of claim 4, wherein the responses comprise a plurality of patient timeline sub-vectors, wherein the patient timeline sub-vectors indicate events relative to a reference date.

13. The computer-implemented method of claim 12, wherein the reference date for a patient timeline sub-vector is the date of birth for a patient associated with the patient timeline sub-vector.

14. The computer-implemented method of claim 4, wherein the responses comprise compressed data table representations of raw patient data, wherein generating the dataset comprises reconstructing a plurality of data tables from the compressed data table representations.

15. The computer-implemented method of claim 14, wherein the reconstructed data tables represent dates as relative dates from a reference date, wherein the reference date is a date of birth of a patient associated with a reconstructed data table, wherein the reconstructed data table is associated with a single patient.

16. A system comprising:
one or more hardware processors; and
a non-transitory computer-readable storage medium having instructions stored thereon that, when executed by the one or more hardware processors, cause the system to:
generate a query, wherein the query includes a plurality of population conditions for defining a cohort for a retrospective medical study;
determine, using one or more feature set matrices, a first set of patients, wherein the first set of patients includes patients who could be included in the cohort based on one or more population conditions of the plurality of population conditions;
generate one or more requests for data from one or more worker nodes, wherein the one or more requests are configured to request data associated with the first set of patients from the one or more worker nodes;
transmit the one or more requests to the one or more worker nodes;
access a plurality of responses from a plurality of worker nodes, the responses generated by the worker nodes in response to executing the one or more generated queries;
generate a dataset based on the plurality of responses, wherein the dataset comprises a plurality of patient timeline vectors for a plurality of cohort patients, wherein at least one of the plurality of patient timeline vectors is generated by combining a first response from a first worker node and a second response from a second worker node that is different from the first worker node.

17. The system of claim 16, wherein the system stores the data from the worker node in volatile memory, wherein the system deletes the data from the volatile memory based on at least one of a completion of a data analysis process or an expiration of a time to live.

18. The system of claim 16, wherein the responses comprise a plurality of patient timeline sub-vectors, wherein the patient timeline sub-vectors indicate events relative to a reference date.

19. The system of claim 16, wherein the responses a plurality of compressed data table representations, wherein the instructions are further configured to cause the system to reconstruct a plurality of data tables from the compressed data table representations.

20. The system of claim 19, wherein the reconstructed data tables represent dates as relative dates from a reference date, wherein the reference data is a date of birth of a patient associated with a reconstructed data table, wherein the reconstructed data table is associated with a single patient.
